(19) United States
(12) Patent Application Publication
(10) Pub. No.: US 2015/0293092 A1
(43) Pub. Date: Oct. 15, 2015

(54) EXTRACELLULAR MATRIX PROTEINS FROM HAEMOPHILUS INFLUENZAE BIOFILMS: TARGETS FOR THERAPEUTIC OR DIAGNOSTIC USE

(71) Applicant: Oak Crest Institute of Science, Pasadena, CA (US)

(72) Inventors: Paul Webster, Pasadena, CA (US); Siva Wu, South Pasadena, CA (US); James Kerwin, Moss Beach, CA (US)

(73) Assignee: OAK CREST INSTITUTE OF SCIENCE, Pasadena, CA (US)

(21) Appl. No.: 14/749,504

(22) Filed: Jun. 24, 2015

Related U.S. Application Data
(63) Continuation of application No. 13/431,592, filed on Mar. 27, 2012, now abandoned.
(60) Provisional application No. 61/468,334, filed on Mar. 28, 2011.

(51) Int. Cl. G01N 33/569; A61K 39/102; C07K 14/285

(52) U.S. Cl. CPC G01N 33/56911; C07K 14/285; A61K 39/102; G01N 2469/10

(57) ABSTRACT

A method of identifying a biofilm that includes non-typeable Haemophilus influenza (NTHi) including a step of screening a sample for the presence of one or more biofilm-specific proteins that are expressed by NTHi. In some cases, an NTHi biofilm-related disease in a subject is diagnosed. Also disclosed are protein microarrays for screening biofilm-specific proteins in a sample, formulations comprising one or more biofilm-specific proteins or fragments thereof, and methods for inducing an immune response in a patient against a biofilm-related infection.

### CROSS-REFERENCE TO RELATED APPLICATIONS

This application claims benefit of U.S. Provisional Application No. 61/468,334 filed Mar. 28, 2011, which is hereby incorporated by reference in its entirety.

### STATEMENT REGARDING FEDERALLY SPONSORED R&D

This invention was made with government support under funds awarded by The National Science Foundation (NSF #0722354) and The National Institute of Deafness and Other Communication Disorders (NIDCD) (5 P-30 DC006276-03). The government has certain rights in the invention.

### BACKGROUND OF THE INVENTION

1. Field of the Invention

A method of identifying a biofilm that comprises non-typeable Haemophilus influenza (NTHi) comprising a step of screening a sample for the presence of one or more biofilm-specific proteins that are expressed by NTHi

2. Description of the Related Art

Otitis media (“OM”) is one of the most common pediatric infectious disorders, second only to the common cold (Bluestone, C. D. & Klein, J. O. (1995) “Clinical practice guideline on otitis media with effusion in young children: strengths and weaknesses.” Otolaryngol Head Neck Surg 112, 507-511) and is also the most common reason for prescribing antibiotics to children in the United States. In the year 2000 the total number of visits to a physician as a result of middle ear infection was estimated to be $16 million, and approximately $13 million antibacterial prescriptions were written (Cherry, D. K. & Woodwell, D. A. (2002) National Ambulatory Medical Care Survey: 2000 summary. Adv Data, 1-32). A course of antibacterial therapy can range in cost from $10 to $100 per individual. If OM were more easily treated, or prevented, the costs of physician visits and treatment would drop considerably. Bouts of OM are usually temporary, yet annual treatment costs were estimated in 1996 to exceed $5 billion (Gates, G. A. (1996) “Cost-effectiveness considerations in otitis media treatment” Otolaryngol Head Neck Surg 114, 525-530).

Dependence on culturing bacterial pathogens has led to gaps in our appreciation of the extent to which these organisms cause disease. It has been estimated that diagnostic laboratories have the ability to identify less than 1% of a bacterial population (Amann R I, et al. “Phylogenetic identification and in situ detection of individual microbial cells without cultivation” Microbiol Rev 1995, 59(1):143-169), and comparisons between culturing and molecular diagnostics only highlight the need for improved diagnostic methods (Dowd S E, et al. 2008 “Survey of bacterial diversity in chronic wounds using pyrosequencing, DGGE, and full ribosome shotgun sequencing” BMC Microbiol 2008, 8:43). The ability of bacteria to form biofilms is one of the main reasons for our lack of success in culturing more species of bacteria (Costerton J W: “Introduction to biofilm” Int J Antimicrob Agents 1999, 11(3-4):217-221; discussion 237-219). For pathogenic bacteria the biofilm phenotype confers an increased resistance to the host immune system as well as increased tolerance to antibiotics and other antimicrobial agents (Hoiby N, et al. “Antibiotic resistance of bacterial biofilms” Int J Antimicrob Agents 2010, 35(4):322-332; Donlan R M and Costerton J W: “Biofilms: survival mechanisms of clinically relevant microorganisms” Clin Microbiol Rev 2002, 15(2):167-193; and Fux C A, et al. “Survival strategies of infectious biofilms” Trends Microbiol 2005, 13(1):34-40). Current antimicrobial therapies have been designed to combat free-swimming, or planktonic bacteria and are not targeted to control bacteria that are present as biofilms (Fux C A, et al. “Bacterial biofilms: a diagnostic and therapeutic challenge” Expert Rev Anti Infect Ther 2003, 1(4):667-683).

Biofilm infections are significantly less susceptible to antibiotics and host-generated immune responses and there are few treatments specifically targeting biofilm infections making them difficult to resolve. Biofilm infections typically manifest as chronic or recurrent infections.

Repeated exposure to antibiotics at an early age might increase susceptibility to asthma and Crohn's disease later in life (Hildebrand, H., et al. (2008) “Early-life exposures associated with antibiotic use and risk of subsequent Crohn's disease” Scand J Gastroenterol 43, 961-966; and Marra, F., et al. (2009) “Antibiotic use in children is associated with increased risk of asthma” Pediatrics 123, 1003-1010).

Currently there is no method for detecting the presence of bacterial biofilms in the middle ear of OM patients. Identifying biofilm formation by non-typeable Haemophilus influenzae (“NTHi”) is desirable because this organism is a common pathogen in upper respiratory tract infections (Murphy, T. F., et al. (2009) “Nontypeable Haemophilus influenzae as a pathogen in children” Pediatr Infect Dis J 28, 43-48) and especially in OM, or middle ear infection (Kerschner, J. E. (2008) “Bench and bedside advances in otitis media” Curr Opin Otolaryngol Head Neck Surg 16, 543-547). Bacterial biofilms may contribute to the pathogenesis of chronic OM, recurrent acute OM, and chronic OM with effusion (Kerschner, J. E. (2008) “Bench and bedside advances in otitis media” Curr Opin Otolaryngol Head Neck Surg 16, 543-547). Treatment of OM consists of oral administration of beta-lactam antibiotics (penicillins and cephalosporins).

### SUMMARY OF THE INVENTION

Some embodiments relate to a method of identifying a biofilm that includes non-typeable Haemophilus influenza (NTHi), including a step of screening a sample for the presence of one or more biofilm-specific proteins that are expressed by NTHi.

In some embodiments, the one or more biofilm-specific proteins are selected from the group consisting of proteins identified by UniProt Accession numbers OPDA_HAEIN, OTCC_HAEIN, 5NTD_HAEIN, DEOD_HAEI8, TKT2_PASMU, ZNUA_HAEIN, FBPA_HAEIN, FBPA_NEIMA; OPP11_HAEIN, Q9KHGO_HAEIN, OPP2A_HAEIN, Q48024_HAEIN, OPP25_HAEIN, OMP51_HAEIN, SIAP_HAEIN, Y362_HAEIN, DNAK2_SYNP6, FKBY_HAEIN, DLDH_HAEIN, G3P_STREQ, G3P_STRP1, KPYK_SPICI and RPOB_HAEI8.

Some embodiments relate to a method for diagnosing an NTHi biofilm-related disease in a subject comprising obtaining a sample from the subject and screening the sample for the presence of one or more biofilm-specific proteins that are expressed by NTHi.

In some embodiments, the NTHi biofilm-related disease includes infection by NTHi in the respiratory tract of the subject.

In some embodiments, the infection by NTHi is in the upper respiratory tract of the subject.

In some embodiments, the NTHi biofilm-related disease includes infection by NTHi in the ear of the subject.

In some embodiments, the disease is Otitis media (OM).

In some embodiments, the sample that is screened is middle ear fluid, saliva or blood.

Some embodiments relate to a protein microarray for screening biofilm-specific proteins in a sample, including a substrate having attached thereto one or more antibodies or fragments thereof, the one or more antibodies or fragments thereof being specific for one or more respective biofilm-specific proteins that are expressed by NTHi.

In some embodiments of protein microarray, the one or more respective biofilm-specific proteins that are expressed by NTHi are selected from the group consisting of proteins identified by UniProt Accession numbers OPDA_HAEIN, OTCC_HAEIN, 5NTD_HAEIN, DEOD_HAEI8, TKT2_PASMU, ZNUA_HAEIN, FBPA_HAEIN, FBPA_NEIMA; OPP11_HAEIN, Q9KHG0_HAEIN, OPP2A_HAEIN, Q48024_HAEIN, OPP25_HAEIN, OMP51_HAEIN, SIAP_HAEIN, Y362_HAEIN, DNAK2_SYNP6, FKBY_HAEIN, DLDH_HAEIN, G3P_STREQ, G3P_STRP1, KPYK_SPICI and RPOB_HAEI8.

Some embodiments relate to a formulation comprising one or more biofilm-specific proteins or fragments thereof, wherein the one or more biofilm-specific proteins are selected from the group consisting of the proteins identified by UniProt Accession numbers OPDA_HAEIN, OTCC_HAEIN, 5NTD_HAEIN, DEOD_HAEI8, TKT2_PASMU, ZNUA_HAEIN, FBPA_HAEIN, FBPA_NEIMA; OPP11_HAEIN, Q9KHG0_HAEIN, OPP2A_HAEIN, Q48024_HAEIN, OPP25_HAEIN, OMP51_HAEIN, SIAP_HAEIN, Y362_HAEIN, DNAK2_SYNP6, FKBY_HAEIN, DLDH_HAEIN, G3P_STREQ, G3P_STRP1, KPYK_SPICI and RPOB_HAEI8.

Some embodiments relate to a method for inducing an immune response in a patient in need thereof against a biofilm-related infection, including administering to the patient a formulation comprising one or more biofilm-specific proteins or fragments thereof.

### BRIEF DESCRIPTION OF THE DRAWINGS

FIG. 1: Scanning EM of aldehyde-fixed NTHi 9274. A. Scanning EM image of a clump of planktonic NTHi bacteria chemically fixed in buffered glutaraldehyde. The bacterial cells (B), a little over 1 μm in length, are bullet shaped with a cell surface covered with uniformly sized small bumps. B. Glutaraldehyde-fixed biofilm bacteria were shorter in length and were associated with large amounts of globular extracellular amorphous material (arrowheads). Scale bars=1 μm.

FIG. 2: Scanning EM of NTHi 9274 colony biofilms. A. Incubation of NTHi bacteria for 1 hr on a support filter results in the filter surface being covered in a layer of amorphous material, which also covers bacteria (arrowheads). B. The support filter with no biofilm shows no evidence of being covered. C. A close view of the 1 hr biofilm bacteria shows bacteria wrapped in amorphous material (arrow), but on top of a second layer of material with embedded bacterial cells (arrowheads). D. The 2 hr biofilm has clumps of bacteria forming under a layer of amorphous material. E. By 12 hr, the biofilm has developed into a mat of tightly packed bacteria with a layer of amorphous material covering the top surface. F. A 4-day biofilm showing how the bacterial cells have arranged themselves into sheets that create pockets of empty space within the biofilm. Scale bars=5 μm.

FIG. 3 Scanning EM of filter surfaces after removal of 1-day and 4-day NTHi biofilms. 1-day and 4-day biofilms were removed from the filter substrates by sonication in PBS and the washed filter surfaces were examined by SEM. A. The washing protocol almost completely removes 1-day biofilms. At low magnification enough of the biofilm material remains on the filter to identify the outer border (arrows) and remnants of the biofilm (arrowhead) within the zone of biofilm formation. Scale bar=500 μm. B. Within the zone of biofilm formation remains a thin network of material attached to the filter surface (F). Scale bar=20 μm. C. Biofilm material remains attached to the surface of filters where 4-day biofilms have been removed using equivalent washing conditions. Scale bar=500 μm. D. The material left behind by the 4-day biofilm resembled a pad of honeycomb-like material. Scale bar=50 μm. Inset. Bacterial cells embedded in the 4-day biofilm residue were more spherical than planktonic NTHi. Scale bar=500 nm.

FIG. 4 Presence of CFUs in washed NTHi 9274 biofilms. Colony biofilms formed on filter substrates were removed by sonication in PBS at increasing time intervals over a 4-day (96 hr) period. A. The numbers of viable bacteria (CFUs) in suspension from each time-point was estimated. Over the 4-day period, the numbers of viable bacteria in the forming biofilms increased 12-fold by 12hr but returned to initial inoculum levels by 4-days. CFUs were 1 hr-5×10⁶; 9 hr-1'10⁹; 12 hr-3×10¹⁰; 15 hr-6×10¹⁰; 24 hr-2×10⁹; 96 hr-7×10⁶. B. The OD₆₀₀ of biofilm material suspended in PBS was used to estimate total biomass in the biofilms. The biofilm biomass peaked at 12 hr and remained at high levels until at the last time point.

FIG. 5 Transmission EM of sections through cryopreserved NTHi 9274 biofilms. A. Low magnification view of a 1-day NTHi biofilm. The top of the biofilm is covered with a layer of amorphous material (arrows). Many cells show no intracellular details but in some there were electron opaque spots located to one pole of the bacterial cell (some examples are labeled with arrowheads). This overview shows the heterogeneity of the bacterial cell profiles and also the absence of obviously damaged cells. Scale bar=1 μm. B. Higher magnification of the 1-day NTHi biofilm shows profiles of irregularly shaped cells. Electron opaque regions are present in some cells (arrowheads), and spaces between cells are filled with ECM (*). Scale bar=500 nm. C. Regions near the base of the 1-day NTHi biofilm and close to the support filter contain large amounts of ECM between the bacterial cells (*). Cryo-preparation methods immobilize this material in situ. Scale bar=500 nm.

FIG. 6. Representative 1D ¹H NMR spectra for water-soluble and water-insoluble fractions.

FIG. 7 Baysian DOSY Transform Spectra.

FIG. 8 HSQC NMR spectra of DMSO-soluble and -insoluble ECM from NTHi 9274 biofilms.

FIG. 9 SDS-PAGE of soluble ECM from 1-day and 4-day biofilms and molecular masses of LC/MS/MS identified proteins from corresponding regions of the gel. The molecular masses of all the proteins identified by proteomic analysis were plotted according to SDS-PAGE band in which they were identified. The 1-day and 4-day proteins were then compared with similar SDS-PAGE gels displayed in the middle of the figure with molecular weight standards (1-d, Std, 4d). Proteins from the 1-day ECM followed an expected pattern. Proteins with lower kDa were found at the bottom of the gel (beginning at band 1), and larger proteins were in the top bands. Proteins from the 4-day ECM showed some high molecular weight proteins identified at the bottom of the gel, and some low molecular mass proteins were detected in the top of the gel. Most proteins still followed the expected pattern within the gel, with low molecular mass proteins at the bottom of the gel and the proteins with higher molecular mass remaining closer to the top of the gel.

FIG. 10 Anti-DNA-directed RNA polymerase antibodies labeling proteins from NTHi 9274 biofilm ECM and biofilm bacteria. The anti-DNA-directed RNA polymerase labels a protein band in the ECM and bacterial pellet from 1-day and 4-day biofilms. The identified protein is band is in the 150 kDa range in all preparations. Weak binding bands in the range of 45-55 kDa (arrowheads) are also present. Western blot showing label on: Lane 1: 1-day biofilm soluble ECM; Lane 2: 1-day biofilm solubilized bacterial pellet; Lane 3: 4-day biofilm soluble ECM; Lane 2: 4-day biofilm solubilized bacterial pellet; kDa: molecular weight markers (kDa).

FIG. 11 DNA-directed RNA polymerase immunolocalization to ECM of 1-day and 4-day NTHi 9274 biofilms. Sections through cryo-preserved NTHi biofilms were labeled with specific antibodies and protein A gold. A. 1-day biofilm. Specific label is associated with the bacterial cells (some of which are labeled “B”) and with ECM (arrows). Scale bar=500 nm. B. 4-day biofilm. Specific label is found over the bacterial profiles (B) as well as ECM around the bacteria (Arrow), and with extracellular vesicles (arrowhead). Scale bar=500 nm. C. 1-day biofilm control. Specific primary antibody was omitted and labeled only with secondary antibody and protein A gold. Some gold particles associate with bacterial profiles (arrowheads) but the labeling density is lower than when primary antibody is included. Scale bar=500 nm. D. 4-day biofilm control labeled only with secondary antibody and protein A gold. Non-specific labeling is detectable over bacterial cells and ECM (arrowheads). The labeling density is lower than when primary antibody is included. Scale bar=500 nm.

FIG. 12 ornithine carbamoyltransferase (OCT) immunolocalization to ECM of 1-day and 4-day NTHi 9274 biofilms. A: Immunolabeling of 1-day colony biofilms show the anti-OCT antibody had low affinity for the biofilm. The label was detected in only low amounts over the extracellular matrix. Most of the label was associated with cell membrane (arrowhead) and within the cell (arrow). Scale bar=100 nm. Insert: Western blot of separated proteins from 1-day (1-d) and 4-day (4-d) NTHi biofilms showing the anti-OCT antibodies binding to two protein bands at 38 kDa (arrowhead) and a lower band at 16 kDa. B: The anti-OCT antibodies labeled 1-day NTHi biofilms formed in capillary tubes. In this image the label is associating with the outer membrane of a bacterium (b) and with a halo of extracellular material (arrowhead) formed around the labeled bacterium. Scale bar=100 nm.

FIG. 13 Pronase disrupts biofilm formation by NTHi 9274 bacteria. A. Pronase causes a decrease in 1-day biofilm formation in crystal violet assays. Bacteria in BHI formed more biofilm than bacteria incubated in BHI containing pronase. The biofilm disrupting effect was concentration dependent, being greatest at higher concentration (1 mg.ml) and decreasing with lower concentrations (0.5 mg/ml or 0.25 mg/ml). B. Scanning EM of part of the outer edge of a 1-day biofilm shows a mat of bacteria with a well-defined border (arrow) running from top left to bottom right of the image. Scale bar=200 μm. C. Scanning EM image of a similar 1-day NTHi biofilm, but on a filter soaked in pronase, shows a mat of NTHi bacteria covering the filter but without a clearly defined biofilm boundary. The edge of the biofilm can be identified (arrow) but is not well defined. Scale bar=200 pm.

FIG. 14. Transketolase immunolocalization outside of bacterial cells of NTHi 9274 biofilms. Antibodies to transketolase were applied to sections of Lowicryl HM20-embedded biofilms formed by non-typeable Haemophilus influenzae (NTHi) bacteria. Four representative are shown (A-D). At the top of the biofilm (A and B) the signal is low but gold particles are associated with extracellular matrix (white arrows) as well as with some bacteria. At the base of the biofilm (C and D) the specific signal is increased over regions of extracellular matrix around and between the bacterial cells. The specific antibody binding is detected by 10 nm size gold particles coupled to protein A, which binds to the specific antibodies bound to the section.

FIG. 15. glyceraldeyhde-3-phospate dehydrogenase (GAPDH) immunolocalization outside of bacterial cells of NTHi 9274 biofilms. (A and B) Antibodies to glyceraldehyde-3-phosphate dehydrogenase (GAPDH) were applied to two sections of Lowicryl HM20-embedded biofilms formed by non-typeable Haemophilus influenzae (NTHi) bacteria. The NTHi (“B”), Gram negative bacilli that are approximately 500 nm in diameter, are embedded in an extracellular matrix (ECM). Antibody binding was detected using 10 nm colloidal gold particles attached to protein A. The gold particles appear as small, black dots on the image, some of which are indicated with arrows over the ECM. Labeling of the ECM was not uniform, with some regions being labeled more than others.

### DETAILED DESCRIPTION OF THE PREFERRED EMBODIMENT

Biofilm infections are difficult to diagnose, primarily because culture methods are not accurate predictors of chronic biofilm infection. Limited demonstration of biofilm infections has been accomplished using minimally invasive techniques (Singh P K, et al. “Quorum-sensing signals indicate that cystic fibrosis lungs are infected with bacterial biofilms” Nature 2000, 407(6805):762-764; and Murphy T F, et al. “Expression of a peroxiredoxin-glutaredoxin by Haemophilus influenzae in biofilms and during human respiratory tract infection” FEMS Immunol Med Microbiol 2005, 44(1):81-89), however, a more rigorous set of diagnostic criteria has been proposed to identify biofilm infections (Parsek M R and Singh P K “Bacterial biofilms: an emerging link to disease pathogenesis” Annu Rev Microbiol 2003, 57:677-701; and Hoiby N, et al. “The clinical impact of bacterial biofilms” Int J Oral Sci 2011, 3(2):55-65). Meeting these criteria requires the demonstration of attached bacteria in an extracellular matrix, which is usually accomplished by microscopy of excised tissues. Microscopy of excised tissues is not a routine protocol in diagnostics so many biofilm infections are not identified during screening. One consequence of our inability to routinely diagnose biofilm infections is our lack of understanding of how widespread they are within the population. Although acceptance of chronic infections caused by biofilms is gradually gaining ground within the research community, their relevance to physicians is less obvious. Less invasive and more direct diagnostic methods for identifying biofilm infections would make it possible to obtain better information on the role of biofilms in chronic infections.

Biofilms are aggregations of sessile bacteria cells embedded in an extracellular polymeric matrix (ECM), which functions to attach bacteria to one another and to biotic and abiotic substrates (Stoodley P, et al. “Biofilms as complex differentiated communities” Annu Rev Microbiol 2002, 56:187-209).

It is a general dogma of biofilm biology that the ECM is composed primarily of exopolysaccharide (EPS) (Sutherland I “Biofilm exopolysaccharides: a strong and sticky framework” Microbiology 2001, 147(Pt 1):3-9). However, analytical examination of a Pseudomonas putida biofilm showed that protein, not polysaccharide, was the largest fraction of extracted polymer (Jahn A, et al. “Composition of Pseudomonas pudita biofilms: Accumulation of protein in the biofilm matrix” Biofouling 1999, 14(1):49-57). A growing interest in applying proteomic analysis to biofilms has reinforced the idea that proteins are a likely biofilm component. Indeed, the number of biofilm specific proteins being identified makes it highly likely that transformation into the biofilm phenotype is a developmental process controlled by selective regulation of protein expression (Vilain S, et al. “Comparative proteomic analysis of planktonic and immobilized Pseudomonas aeruginosa cells: a multivariate statistical approach” Anal Biochem 2004, 329(1):120-130; Sauer K and Camper A K “Characterization of phenotypic changes in Pseudomonas putida in response to surface-associated growth” J Bacteriol 2001, 183(22):6579-6589; Sauer K, et al. “Pseudomonas aeruginosa displays multiple phenotypes during development as a biofilm” J Bacteriol 2002, 184(4):1140-1154; Allegrucci M, et al. “Phenotypic characterization of Streptococcus pneumoniae biofilm development” J Bacteriol 2006, 188(7):2325-2335; Oosthuizen M C, et al. “Proteomic analysis reveals differential protein expression by Bacillus cereus during biofilm formation” Appl Environ Microbiol 2002, 68(6):2770-2780; and Southey-Pillig C J, et al. “Characterization of temporal protein production in Pseudomonas aeruginosa biofilms” J Bacteriol 2005, 187(23):8114-8126). Protein driven mechanisms for the shift to a biofilm phenotype could become targets for disrupting the biofilm-forming process.

Depending on the species of bacteria and growth conditions, the ECM is composed of varying amounts of exopolysaccharides, nucleic acids, lipids and proteins (Flemming H C and Wingender J “Relevance of microbial extracellular polymeric substances (EPSs)—Part I: Structural and ecological aspects” Water Sci Technol 2001, 43(6):1-8). Proteins in the ECM may derive from lysed cells or be actively secreted by biofilm-forming bacteria (Hoffman M and Decho A W “Extracellular enzymes within microbial biofilms and the role of the extracellular polymer matrix” In: Microbial Extracellular Polymeric Substances. Edited by Wingender J, Neu J, Flemming H-C. Berlin, Germany: Springer-Verlag; 1999). Extracellular proteins can be firmly bound to the cell surface, associated with the ECM, or freely diffusing throughout the biofilm matrix (Hoffman M and Decho A W “Extracellular enzymes within microbial biofilms and the role of the extracellular polymer matrix” In: Microbial Extracellular Polymeric Substances. Edited by Wingender J, Neu J, Flemming H-C. Berlin, Germany: Springer-Verlag; 1999). The presence and function of extracellular proteins has been reported for a wide range of Gram-positive and Gram-negative bacteria, both in cultures of free-swimming (planktonic) cells and in biofilms (Wingender J, et al. “Interaction between extracellular polysaccharides and enyzmes” In: Microbial Extracellular Polymeric Substances. Edited by Wingender J, Neu J, Flemming H-C. Berlin, Germany: Springer-Verlag; 1999). Extracellular proteins from planktonic cells are present in low concentration and are a minor component of a complex in vivo milieu. However, the confined microenvironment of the biofilm matrix traps extracellular molecules and prolongs the stability and activity of extracellular enzymes (Hoffman M and Decho A W “Extracellular enzymes within microbial biofilms and the role of the extracellular polymer matrix” In: Microbial Extracellular Polymeric Substances. Edited by Wingender J, Neu J, Flemming H-C. Berlin, Germany: Springer-Verlag; 1999). The variety, abundance and distribution of extracellular proteins within the ECM of biofilms have attracted only minimal interest within the scientific community (Southey-Pillig C J, et al. “Characterization of temporal protein production in Pseudomonas aeruginosa biofilms” J Bacteriol 2005, 187(23):8114-8126; and Webster P, et al. “Distribution of bacterial proteins in biofilms formed by non-typeable Haemophilus influenzae. J Histochem Cytochem 2006), yet, these proteins may contribute to inflammatory responses observed in chronic infections. Proteins within the ECM may also assist in forming the complex structures reported within biofilms (Allegrucci M, et al. “Phenotypic characterization of Streptococcus pneumoniae biofilm development” J Bacteriol 2006, 188(7):2325-2335; and Oosthuizen M C, et al. “Proteomic analysis reveals differential protein expression by Bacillus cereus during biofilm formation” Appl Environ Microbiol 2002, 68(6):2770-2780). Soluble proteins uniquely expressed by biofilms may be useful as quantitative diagnostic biomarkers.

Nontypeable Haemophilus influenzae is a significant pathogen in children, causing otitis media (OM), sinusitis, conjunctivitis, pneumonia, and occasionally invasive infections. Approximately, one-third of episodes of OM are caused by nontypeable H. influenzae and the bacterium is the most common cause of recurrent otitis media.

Here we examine the early stages of in vitro biofilm formation by non-typeable Haemophilus influenzae (NTHi) to determine if it is possible to detect biofilm-specific proteins in soluble fractions of the ECM. NTHi are Gram-negative commensal organisms of humans that colonize the respiratory tract and can act as opportunistic pathogens. NTHi have been implicated in sinus infections and the organism is a major pathogen of otitis media or middle ear infections (Hall-Stoodley L, et al “Direct detection of bacterial biofilms on the middle-ear mucosa of children with chronic otitis media” Jama 2006, 296(2):202-211; and Barkai G, et al. “Potential contribution by nontypable Haemophilus influenzae in protracted and recurrent acute otitis media” Pediatr Infect Dis J 2009, 28(6):466-471). NTHi is also linked to infections of the lungs of cystic fibrosis patients (Starner T D et al. “Haemophilus influenzae forms biofilms on airway epithelia: implications in cystic fibrosis” Am J Respir Crit Care Med 2006, 174(2):213-220), and morbidity in patients with chronic obstructive pulmonary disease (COPD) (Murphy T F et al. “Persistent colonization by Haemophilus influenzae in chronic obstructive pulmonary disease. Am J Respir Crit Care Med 2004, 170(3):266-272; Thanavala Y and Lugade A A “Role of nontypeable Haemophilus influenzae in otitis media and chronic obstructive pulmonary disease” Adv Otorhinolaryngol 2011, 72:170-175; and Moghaddam S J, et al. “Nontypeable Haemophilus influenzae in chronic obstructive pulmonary disease and lung cancer” Int J Chron Obstruct Pulmon Dis 2011, 6:113-123). NTHi is a demonstrated biofilm-forming organism when present in vivo causing infection (Murphy T F, et al. “Expression of a peroxiredoxin-glutaredoxin by Haemophilus influenzae in biofilms and during human respiratory tract infection” FEMS Immunol Med Microbiol 2005, 44(1):81-89; Hall-Stoodley L, et al “Direct detection of bacterial biofilms on the middle-ear mucosa of children with chronic otitis media” Jama 2006, 296(2):202-211; and Murphy T F et al. “Nontypeable Haemophilus influenzae as a pathogen in children” Pediatr Infect Dis J 2009, 28(1):43-48) in animal model systems (Jurcisek J et al. “Role of sialic acid and complex carbohydrate biosynthesis in biofilm formation by nontypeable Haemophilus influenzae in the chinchilla middle ear.” Infect Immun 2005, 73(6):3210-3218; Jurcisek J A, et al. “Anatomy of the nasal cavity in the chinchilla” Cells Tissues Organs 2003, 174(3):136-152; and Miyamoto N and Bakaletz L O “Selective adherence of non-typeable Haemophilus influenzae (NTHi) to mucus or epithelial cells in the chinchilla eustachian tube and middle ear” Microb Pathog 1996, 21(5):343-356), and in vitro (Webster P, et al. “Distribution of bacterial proteins in biofilms formed by non-typeable Haemophilus influenzae. J Histochem Cytochem 2006, Murphy T F and Kirkham C “Biofilm formation by nontypeable Haemophilus influenzae: strain variability, outer membrane antigen expression and role of pili” BMC Microbiol 2002, 2:7; Webster P, et al. “Ultrastructural preservation of biofilms formed by non-typeable Hemophilus influenzae” Biofilms 2004, 1:165-182; Swords W E, et al. “Sialylation of lipooligosaccharides promotes biofilm formation by nontypeable Haemophilus influenzae” Infect Immun 2004, 72(1):106-113; and Moriyama S, et al. “Formation of biofilm by Haemophilus influenzae isolated from pediatric intractable otitis media” Auris Nasus Larynx 2009).

The concentration and complexity of extracellular polymeric matrices associated with biofilms in vitro and in vivo may have been underestimated because standard preparation methods to preserve these fragile structures remove soluble components before characterization (Webster P, et al. “Ultrastructural preservation of biofilms formed by non-typeable Hemophilus influenzae” Biofilms 2004, 1:165-182). In our previous studies we have documented the presence of large amounts of proteins in the NTHi ECM using immunocytochemistry (Webster P, et al. “Distribution of bacterial proteins in biofilms formed by non-typeable Haemophilus influenzae. J Histochem Cytochem 2006) and proteomic analysis (Gallaher T K, et al. “Identification of biofilm proteins in non-typeable Haemophilus influenzae” BMC Microbiol 2006, 6:65). In the current study we examine the soluble portions of NTHi ECM using Fourier transform infrared spectroscopy (FTIR), nuclear magnetic resonance spectroscopy (NMR), chemical analysis, and mass spectroscopy. We use cryo-preparation methods to preserve the fragile ECM of in vitro biofilms formed by NTHi bacteria and immunolocalize, DNA-directed RNA polymerase, a biofilm-specific protein in the ECM. DNA-directed RNA polymerase is considered to have a cytoplasmic location, prompting the question of how this protein is delivered to the ECM.

We based our research on the hypothesis that biofilms formed by non-typeable Haemophilus influenzae (NTHi) release unique proteins into middle ear fluids and into the saliva of patients with OM, sinus infection and chronic obstructive pulmonary disease (COPD). The presence of these unique proteins can be used as biomarkers to diagnose the presence of an NTHi biofilm during an infection. At the present time, there are no diagnostic tests available that will detect the biofilm phenotype for any bacterial species. These biofilm-specific proteins can also be used as therapeutic targets.

We have identified 18 unique proteins released from NTHi biofilms formed in vitro, which provide a basis to screen middle ear fluids removed from the middle ears of patients with OM (Table 1). Middle ear fluid collection is performed by conventional techniques.

The presence of biofilm-specific proteins makes it possible to identify biofilm structures in the middle ear of OM patients. It is possible that the biofilm-specific proteins are also involved with formation and maintenance of the biofilm. Thus, biofilm-specific proteins may be incorporated into NTHi biofilm-specific vaccines.

Biofilm-specific proteins have been examined in preparations from biofilms formed by other species of bacteria. There is very little information available on the use of the extracellular matrix by bacteria to assemble a biofilm. The proteins we have identified have not previously been studied in the context of biofilm formation by NTHi bacteria. Understanding the composition of biofilms formed by a bacterial pathogen and how the biofilm is assembled will promote development of new treatment options for recalcitrant infections. Information on the proteins that are linked with bacterial biofilms will make it possible to develop diagnostic tests that can be used by physicians in their office.

Currently the most elaborate screening that is performed on middle ear fluids is to perform culture analysis. However, many species of biofilm-forming bacteria cannot be cultured in the microbiology laboratory. Culturing middle ear fluids often produces negative results, and this may have misled researchers into thinking that bacteria did not play a role in middle ear infections.

Other vaccine candidates have been based on proteins isolated from free-swimming (planktonic) forms of NTHi (Berenson, C. S., et al. (2005) “Outer membrane protein P6 of nontypeable Haemophilus influenzae is a potent and selective inducer of human macrophage proinflammatory cytokines” Infect Immun 73, 2728-2735; Chang, A., et al. (2010) “Haemophilus influenzae outer membrane protein P6 molecular characterization may not differentiate all strains of H. Influenzae from H. haemolyticus” J Clin Microbiol 48, 3756-3757; Chang, A., et al. (2011) “Haemophilus influenzae vaccine candidate outer membrane protein P6 is not conserved in all strains” Hum Vaccin 7; Mes, T. H. & van Putten, J. P. (2007) “Positively selected codons in immune-exposed loops of the vaccine candidate OMP-P1 of Haemophilus influenzae” J Mol Evol 64, 411-422; Novotny, L. A., et al. (2002) “Detection and characterization of pediatric serum antibody to the OMP P5-homologous adhesin of nontypeable Haemophilus influenzae during acute otitis media” Vaccine 20, 3590-3597; Novotny, L. A., et al. (2009) “Epitope mapping immunodominant regions of the PilA protein of nontypeable Haemophilus influenzae (NTHI) to facilitate the design of two novel chimeric vaccine candidates” Vaccine 28, 279-289; Ostberg, K. L., et al. (2009) “Mucosal immunization of mice with recombinant OMP P2 induces antibodies that bind to surface epitopes of multiple strains of nontypeable Haemophilus influenzae.” Mucosal Immunol 2, 63-73; and Riedmann, E. M., et al. (2003) “Construction of recombinant S-layer proteins (rSbsA) and their expression in bacterial ghosts—a delivery system for the nontypeable Haemophilus influenzae antigen Omp26” FEMS Immunol Med Microbiol 37, 185-192).

Our hypothesis is that biofilm formation is a developmental process where biofilm-specific proteins are expressed. We are examining the role of the biofilm-specific proteins in establishment and maintenance of the biofilm. However, if some of these biofilm-specific proteins are soluble and are released into the surrounding environment, such as the middle ear, or in saliva, it will be possible to design a simple detection assay.

If the biofilm-unique proteins are involved in maintenance of the biofilm phenotype, then antibodies that specifically target these proteins can be used to disrupt already formed biofilms.

A diagnostic test for bacterial biofilms will be unique. Using biofilm proteins as a basis for vaccine therapy is also unique. In order to continue the development of a diagnostic test, and identify therapeutic targets, we will screen middle ear fluids from children with OM. The children will be taken from a population of patients who attend the otolaryngology department of the Childrens Hospital Los Angeles.

If the proteins we identified are unique to NTHi biofilms, we will be able to develop antibody-based, compact diagnostic kits that can be sold at low cost to pediatric physicians and otolaryngology departments in local and regional hospitals.

### Protein Screening Methods

There are many methods known in the art for protein identification. These include immunoassay methods, which rely on an antibody or antibody fragment for specifically recognizing and binding to a protein target. Such immunoassays include ELISA (enzyme-linked immuno-sandwich assay) and variations of enzyme-linked detection of antibody binding, radioimmunoassay and Western blotting. Also, included are protein microarray detection methods, wherein antibodies or fragments thereof are spotted on a solid substrate, followed by contacting samples, washing to remove unbound proteins and then detection using a variety of well-known detection chemistries. Other methods for identifying proteins in a sample include Edman sequencing, mass spectrometry, including matrix-assisted laser desorption/ionization mass spectrometry (MALDI-MS) (including peptide mass fingerprinting and post-source decay analysis); see e.g., Kris Gevaert et al., Electrophroresis 2000, 21; 1145-1154; McHugh L, Arthur J W, 2008 Computational Methods for Protein Identification from Mass Spectrometry Data. PLoS Comput Biol 4(2): e12. doi:10.1371/journal.pcbi.0040012; incorporated in their entirety herein by reference.

### Antibodies

The term “antibody” as used herein includes intact immunoglobulin molecules, as well as fragments thereof, such as Fab, F(ab′)₂, and Fv, which are capable of binding an epitope of a protein. Typically, at least 6, 8, 10, or 12 contiguous amino acids are required to form an epitope. However, epitopes that involve non-contiguous amino acids may require more, e.g., at least 15, 25, or 50 amino acid. An antibody that specifically binds to an epitope of a protein of interest can be used therapeutically, as well as in immunochemical assays, such as Western blots, ELISAs, radio immunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the protein of interest.

Typically, an antibody that specifically binds to a protein of interest provides a detection signal at least 5-, 10-, or 20-fold higher than a detection signal provided with other proteins when used in an immunochemical assay. Preferably, antibodies that specifically bind to a protein of interest do not detect other proteins in immunochemical assays and can immunoprecipitate the protein of interest from solution.

The proteins identified herein can be used to immunize a mammal, such as a mouse, rat or rabbit to produce polyclonal antibodies. If desired, a protein of interest can be conjugated to a carrier protein, such as bovine serum albumin, thyroglobulin, and keyhole limpet hemocyanin. Depending on the host species, various adjuvants can be used to increase the immunological response.

Monoclonal antibodies that specifically bind to a protein of interest can be prepared using any technique that provides for the production of antibody molecules by continuous cell lines in culture. These techniques include, but are not limited to, the hybridoma technique, the human B-cell hybridoma technique, and the EBV-hybridoma technique.

In addition, techniques developed for the production of “chimeric antibodies”, the splicing of mouse antibody genes to human antibody genes to obtain a molecule with appropriate antigen specificity and biological activity, can be used. Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues that differ from those in the human sequences by site directed mutagenesis of individual residues or by grafting of entire complementarily determining regions. Antibodies that specifically bind to a protein of interest can contain antigen binding sites that are either partially or fully humanized.

Alternatively, techniques described for the production of single chain antibodies can be adapted using methods known in the art to produce single chain antibodies that specifically bind to a protein of interest. Antibodies with related specificity, but of distinct idiotypic composition, can be generated by chain shuffling from random combinatorial immunoglobin libraries. Single-chain antibodies also can be constructed using a DNA amplification method, such as PCR, using hybridoma cDNA as a template. Single-chain antibodies can be mono- or bispecific, and can be bivalent or tetravalent. Construction of tetravalent, bispecific single-chain antibodies is taught. A nucleotide sequence encoding a single-chain antibody can be constructed using manual or automated nucleotide synthesis, cloned into an expression construct using standard recombinant DNA methods, and introduced into a cell to express the coding sequence. Alternatively, single-chain antibodies can be produced directly using, for example, filamentous phage technology.

Antibodies that specifically bind to a protein of interest also can be produced by inducing in vivo production in the lymphocyte population or by screening immunoglobulin libraries or panels of highly specific binding reagents. Other types of antibodies can be constructed and used therapeutically in methods of the invention. Binding proteins that are derived from immunoglobulins and that are multivalent and multispecific also can be prepared.

Antibodies according to the invention can be purified by methods well known in the art. For example, antibodies can be affinity purified by passage over a column to which a protein of interest is bound. The bound antibodies can then be eluted from the column using a buffer with a high salt concentration.

### Vaccine Formulations

In another aspect, vaccine formulations and methods of inducing immunity are disclosed herein. Any one or more of the biofilm-specific proteins or fragments thereof may be used in formulating a vaccine against biofilm-related OM as well as other biofilm-related clinical indications. Vaccine formulation techniques are well know. See e.g., Morefield, A Rational, Systematic Approach for the Development of Vaccine Formulations, AAPS Journal, 2011 10.1208/s12248-011-9261-1; incorporated in its entirety herein by reference.

### Definitions

The term “biofilm” refers to a complex structure adhering to surfaces that are regularly in contact with water, consisting of colonies of bacteria and usually other microorganisms such as yeasts, fungi, and protozoa that secrete a mucilaginous protective coating in which they are encased. Biofilms typically comprise microorganisms that are embedded in extracellular matrices or polymers. Biofilms can form on solid or liquid surfaces as well as on soft tissue in living organisms, and are typically resistant to conventional methods of disinfection. Biofilms are generally pathogenic in the body, causing such diseases as cystic fibrosis and otitis media.

As used herein, the phrase “inducing an immune response in a subject” is a term understood in the art and refers to an increase of at least about 2-fold, or alternatively at least about 5-fold, or alternatively at least about 10-fold, or alternatively at least about 100-fold, or alternatively at least about 500-fold, or alternatively at least about 1000-fold or more in an immune response to an antigen (or epitope) which can be detected or measured, after introducing the antigen (or epitope) into the subject, relative to the immune response (if any) before introduction of the antigen (or epitope) into the subject. An immune response to an antigen (or epitope), includes but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen-specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody).

“Adjuvants” are well known in the art. Adjuvants useful with the present invention include MoGM-CSF, TiterMax-G, CRL-I005, GERBU, TERamide, PSC97B, Adjumer, PG-026, GSK-1, GcMAF, B-alethine, MPC-026, Adjuvax, CpG ODN, Betafectin, Alum, AS04 and MF59 (see Kim et al. Vaccine 1999 18:597-603 and references therein).

Other adjuvants include, for example, chemokines (e.g., defensins, HCC-1, HCC4, MCP-1, MCP-3, MCP4, MIP-1α, MIP-1β, MIP-1δ, MIP-3α, MIP-2, RANTES); other ligands of chemokine receptors (e.g., CCR1, CCR-2, CCR-5, CCR-6, CXCR-1); cytokines (e.g., IL-1β, IL-2, IL-6, IL-8, IL-10, IL-12; IFN-γ; TNF-α; GM-CSF); other ligands of receptors for those cytokines, AGP, AS02, DC-Chol, Detox, OM-174); ISCOMS and saponins (e.g., QUIL A, QS-21); squalene; superantigens; or salts (e.g., aluminum hydroxide or phosphate, calcium phosphate). In preferred embodiments, an adjuvant does not contain bacterial protein(s). In other preferred embodiments, an adjuvant is a synthetic adjuvant.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

### EXAMPLE

### Biofilm-Specific Extracellular Matrix Proteins of Non-Typeable Haemophilus Influenzae

Bacteria often proliferate as biofilms, embedded in a complex extracellular polymeric matrix (ECM). Non-typeable Haemophilus influenzae (NTHi) is a biofilm-forming organism that causes upper and lower respiratory tract infections such as otitis media, chronic sinus infections and COPD. In this study we examine proteins from the ECM of NTHi biofilms to determine if there are easily detectable, biofilm-specific proteins that could be used for NTHi biofilm diagnosis.

One- and four-day old NTHi biofilms contained bacterial cells enclosed in a thin layer of ECM. The ECM material could be separated from bacterial cells for analysis. NMR and FTIR analysis of the ECM detected polysaccharides and proteinaceous components. Eighteen biofilm-specific proteins were identified in the NTHi biofilm ECM using liquid chromatography-tandem mass spectroscopy. One protein was unique to 1-day biofilms, two were found only in 4-day biofilms, and fifteen were present at both 1-day and 4-day NTHi biofilms. All proteins identified were either associated with bacterial membranes or were cytoplasmic proteins involved with metabolism, transcription or protein folding. One protein, a DNA-directed RNA polymerase, was detected by immunocytochemistry in bacterial cells within the biofilms and in the ECM.

Release of cytoplasmic proteins may be involved in the in vitro formation of NTHi biofilms. 18 biofilm-specific proteins were identified from soluble ECM, one of which was located in the ECM using specific antibodies. Although extreme degradation of bacterial cells was not detected morphologically, cytoplasmic proteins are a component of the extracellular matrix of newly forming NTHi biofilms. Easily solubilized, biofilm-specific proteins might offer a unique approach for detecting NTHi biofilms in vivo. If such a diagnostic tool were quantifiable it could open up new ways of identifying biofim infections and evaluating the extent of biofilm infections in the population.

### The ECM of NTHi Biofilms

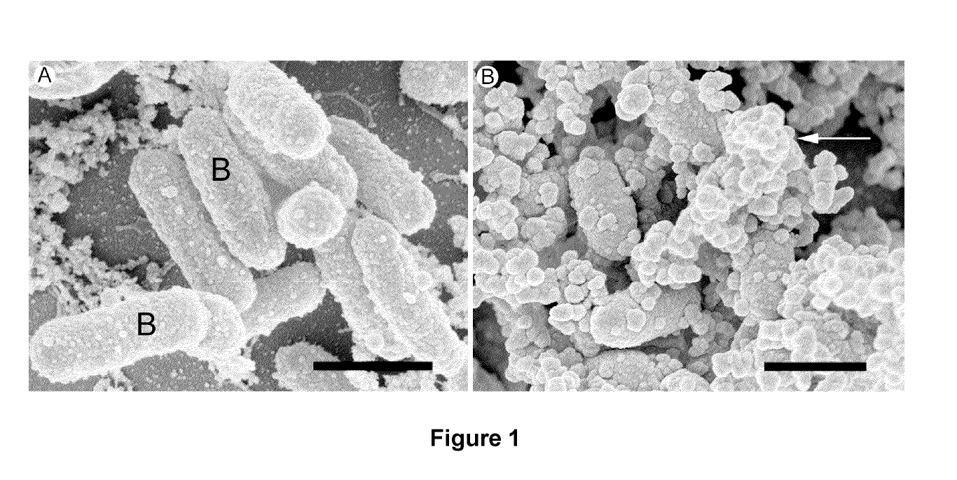

Pellets of planktonic forms of NTHi bacteria mounted onto a filter substrate and then chemically fixed by immersion in buffered glutaraldehyde, showed a classical appearance in the SEM (FIG. 1A). The bacteria were bullet-shaped cells approximately 0.5 μm in length, with an outer surface covered in a uniform layer of small bumps (FIG. 1A). However, if the bacteria were left overnight to form biofilms on the filter substrate before chemical fixation, the fixed biofilm bacteria had an altered shape and size, with beads of extracellular material attached to their surface (FIG. 1B).

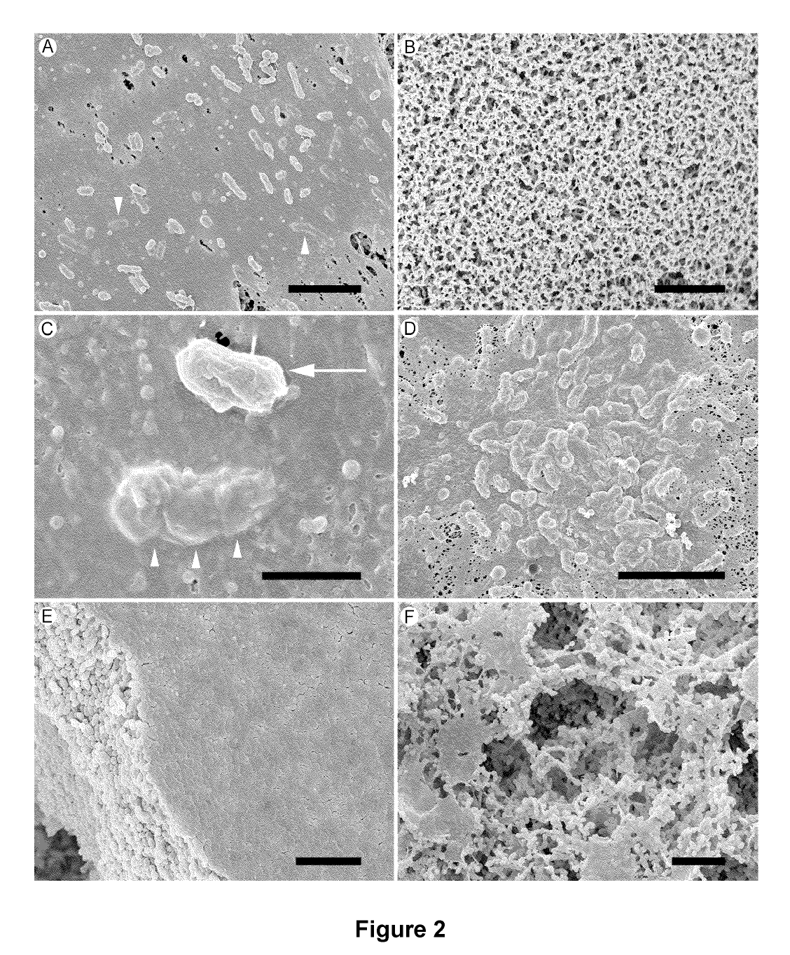

By contrast, diluted suspensions of NTHi bacteria incubated on filters and then prepared for scanning electron microscopy (SEM) examination using cryomethods showed an altered morphology. Bacteria inoculated on filters form colony biofilms over time, a process that can be observed using SEM. After 1 hr incubation the filter surfaces were covered with extensive amounts of amorphous material (FIG. 2A). Similar filters that had not been inoculated with NTHi suspensions were not covered with this amorphous material (FIG. 2B). The amorphous material wrapped the bacterial cells and also covered them (FIG. 2C). By 2 hr incubation, bacteria on the filter surface were completely covered with amorphous material and appeared to be forming clumps of increased numbers of cells, suggesting cell division was occurring (FIG. 2D). 6 hr incubation produced a dense biofilm of tightly packed bacterial cells, which were similar in appearance to the tightly packed biofilms formed after 12 hr incubation (FIG. 2E), and were also similar in cell packing and thickness to the biofilms formed after 24 hr. When left for 96 hr (4-days) without further feeding, the cells in the biofilms became less densely packed, and the bacteria were arranged into thin sheets to form empty pockets (FIG. 2F).

The 1-day and 4-day colony biofilms that formed on the surface of the 22 mm diameter membrane filters remained within the area inoculated with NTHi suspension with no observable spreading of bacteria outside of this area. The mean surface area of the biofilms was 380 mm² and the mean total biomass of the 1-day and 4-day biofilms were 5,590 and 5,810 μg/biofilm respectively (Table 2). In order to collect the soluble components of the bacterial ECM for analysis we deliberately washed the colony biofilms by sonication in PBS. Soluble protein content of the washed suspensions after insoluble material had been removed by centrifugation was 220 μg/biofilm for 1-day biofilms and 194 μg/biofilm for the 4-day biofilms. The total extractable protein obtained from the ECM of each 1-day biofilm was 559 μg, and 483 μg from each 4-day biofilm (Table 2). DNA content of the soluble ECM from 1-day biofilm was 6.8 μg and 7.6 μg from the ECM of the 4-day biofilm.

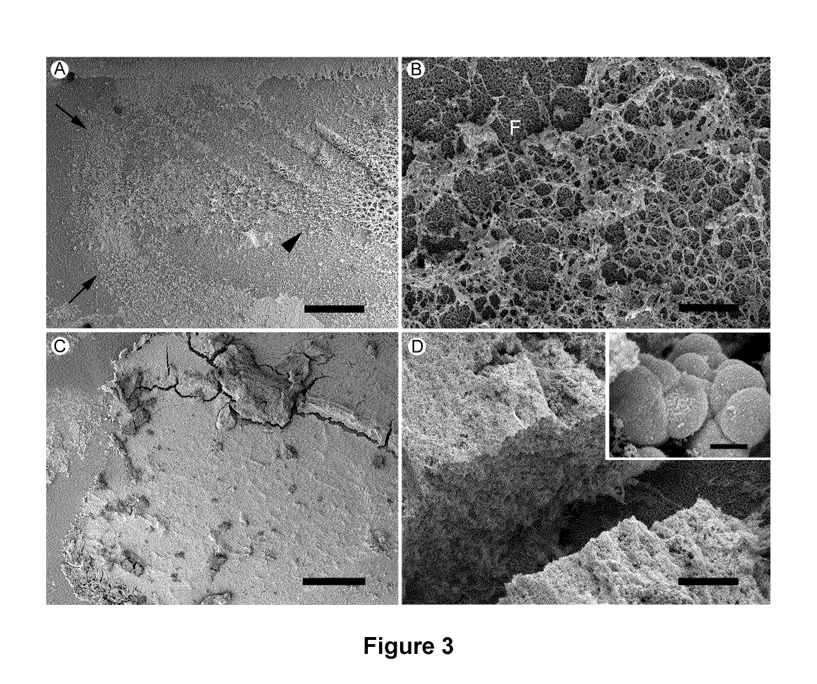

The washing protocol efficiently removed 1-day biofilms from the filter substrates leaving only a thin lattice network of material attached to the filter (FIGS. 3A & C), and no detectable bacterial cells. The washing protocol was not as efficient at removing 4-day biofilms, and left large amounts of attached residual biofilm material (FIG. 3B & D) which consisted of small numbers of bacterial cells embedded in honeycomb-like material (FIGS. 3C & D). The NTHi bacteria that remained attached to the 4-day washed filters were not typical for NTHi in that the cells were more spherical in shape (FIG. 3D insert).

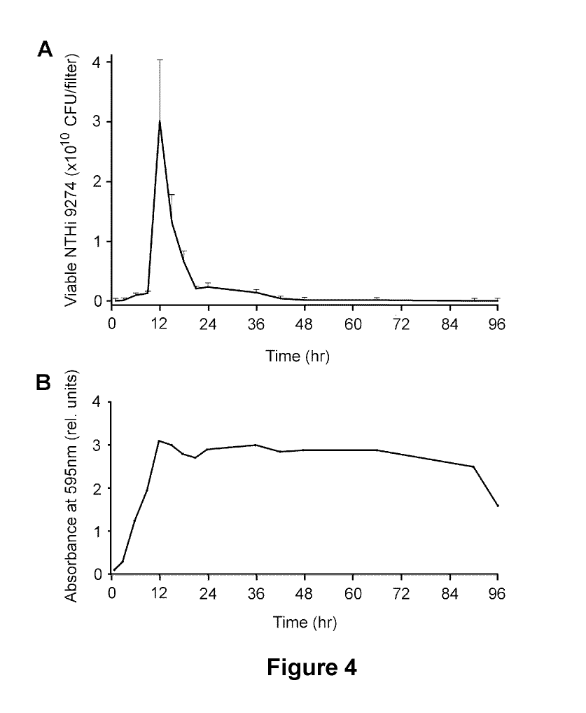

To determine if the washing protocol was damaging the bacterial cells in the biofilms we cultured the solubilized biofilm material removed from the filters and obtaining estimates of viable NTHi in forming biofilms (as CFUs per filter) over a 4-day period (FIG. 4A). During the early time points the numbers of viable bacteria slowly increased from approximately 6.1×10⁶ at 1 hr after incubation, to 1.3×10⁹ after 9 hrs (FIG. 4A). Between 9 hr and 12 hr, the numbers of viable bacteria increased rapidly until by 12 hr, there were approximately 3.0×10¹⁰ NTHi per biofilm. When examined again at 15 hr, the numbers of viable bacteria per biofilm had dropped to approx 1.3×10¹⁰. The numbers of viable NTHi continued to fall until at 24 hr, they had dropped to 2.3×10⁹, a level similar to that observed after 9 hr incubation. Viable bacteria per biofilm continued to fall until at 96 hr there were a mean number of 8.0×10⁶ NTHi present per biofilm. The number of viable bacteria present in the 4-day biofilm (8.0×10⁶) was greater than 1 hr biofilms (6.1×10⁶); however, 4-day biofilms contained fewer culturable bacteria 8.0×10⁶ than were present in the 24 hr biofilm (2.3×10⁹).

The OD₆₀₀ of the total bacterial suspensions washed from the support filters was used as a rough estimate of the biomass washed from the filter at increasing time points (FIG. 4B). The total biomass peaked at 12 hr, corresponding to the peak in viable bacteria (FIG. 4B). However the biomass increase began before any large increase in the numbers of viable bacteria was observed (FIG. 4B). This result suggests that production of extracellular matrix occurs before proliferation of bacterial cells.

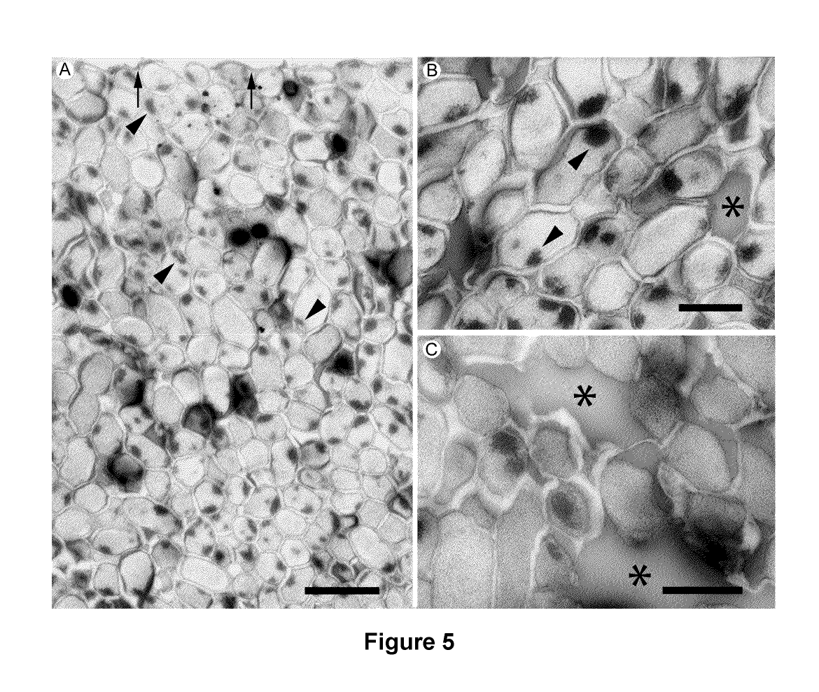

In the transmission electron microscope (TEM), cryoprepared 1-day colony biofilms were composed of densely packed bacteria, heterogeneous in size and shape, and with few intracellular structures (FIG. 5). A layer of extracellular material covered the top of the biofilm (FIG. 5A) and ECM was observed between cells in the middle (FIG. 5B) and base of the biofilm (FIG. 5C). Although most of the NTHi were devoid of intracellular detail (FIG. 5), some bacterial cells contained small dark regions (FIG. 5). Sections through the whole biofilm gave an estimate of 16 μm for the mean height of the 24 hr biofilms formed by the NTHi.

### Chemical Analysis, FTIR and NMR

IR spectra of Day 1 and Day 4 water-insoluble biofilms were indistinguishable, but significantly different from the corresponding water-soluble fractions. The main spectral bands for these samples are shown and assigned in Tables 3 and 4. The water-soluble biofilms seemed to contain a mixture of polysaccharides and proteinaceous material, while the water-insoluble fractions of the biofilms consisted largely of proteinaceous material, with the 1160 and 1084 cm⁻¹ bands characteristic of polysaccharides being absent.

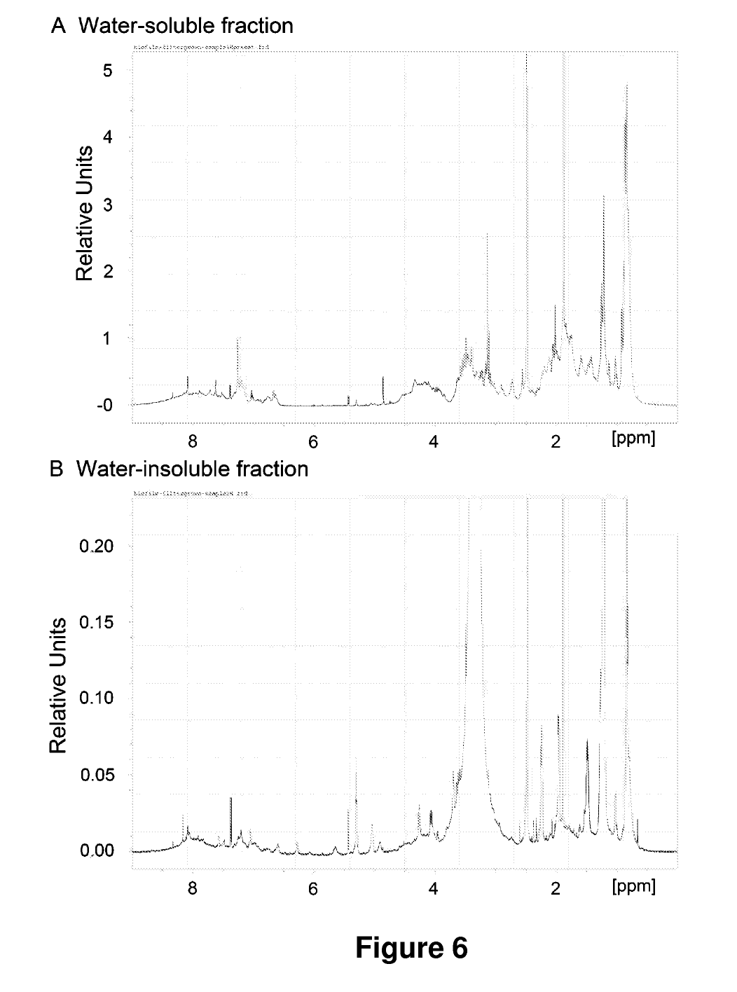

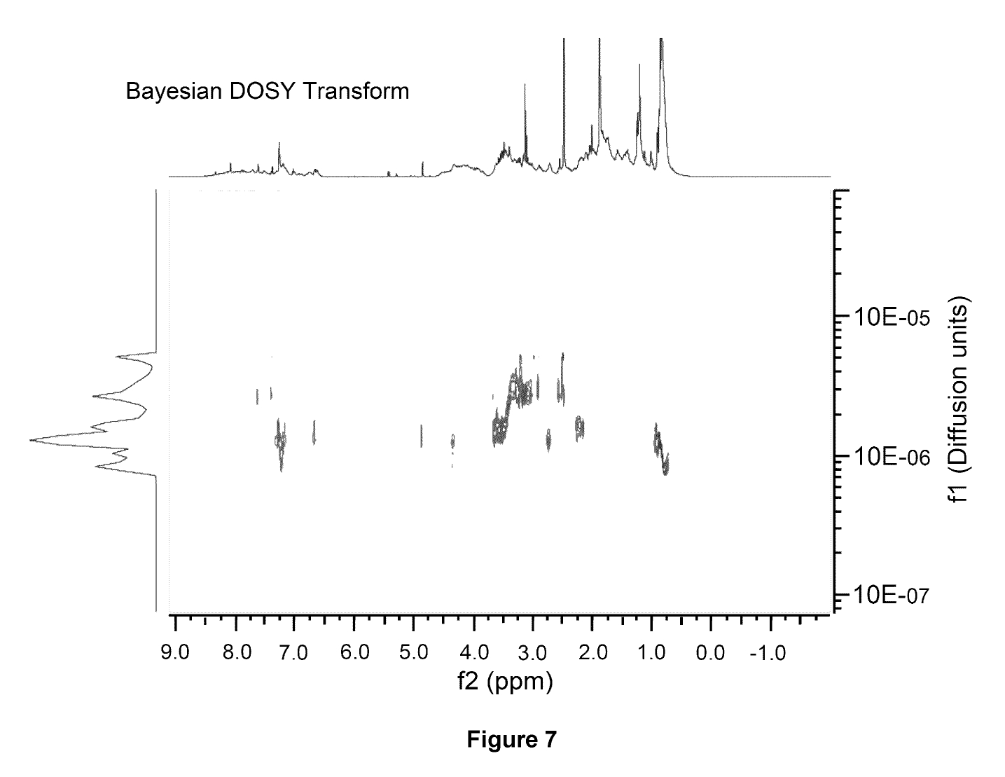

NMR spectra were obtained using DMSO-soluble biofilm samples, representing the major portion of the material (>75%). The fractions were poorly water- and trifluoroacetic acid-soluble. Aqueous acids were not used to solubilize the material as this would depolymerize a range of macromolecular biomolecules. The water-soluble and -insoluble biofilm fractions obtained at Day 1 and Day 4 produced similar ¹H NMR spectra, respectively. The unchanged chemical composition over the four days of incubation supports observations using FTIR spectroscopy. Representative 1D ¹H spectra are presented in FIG. 6. ¹H spectra of the water-soluble fraction (FIG. 6A) contained numerous peaks with narrow line-widths, suggesting the presence of small molecules (i.e., not biopolymers), in addition to numerous peaks in the 3.0-4.5 ppm range. These features were less abundant in ¹H spectra of the water-insoluble material (FIG. 6B). The above observations are supported by DOSY spectra, which show a significant distribution of molecular masses in the water-soluble samples (FIG. 7), while the water-insoluble fractions did not. The DOSY spectrum (FIG. 7) appears to show the presence of lower molecular weight saccharides (d_{H} 2.5 and 2.75-3.5 ppm), as well as proteins.

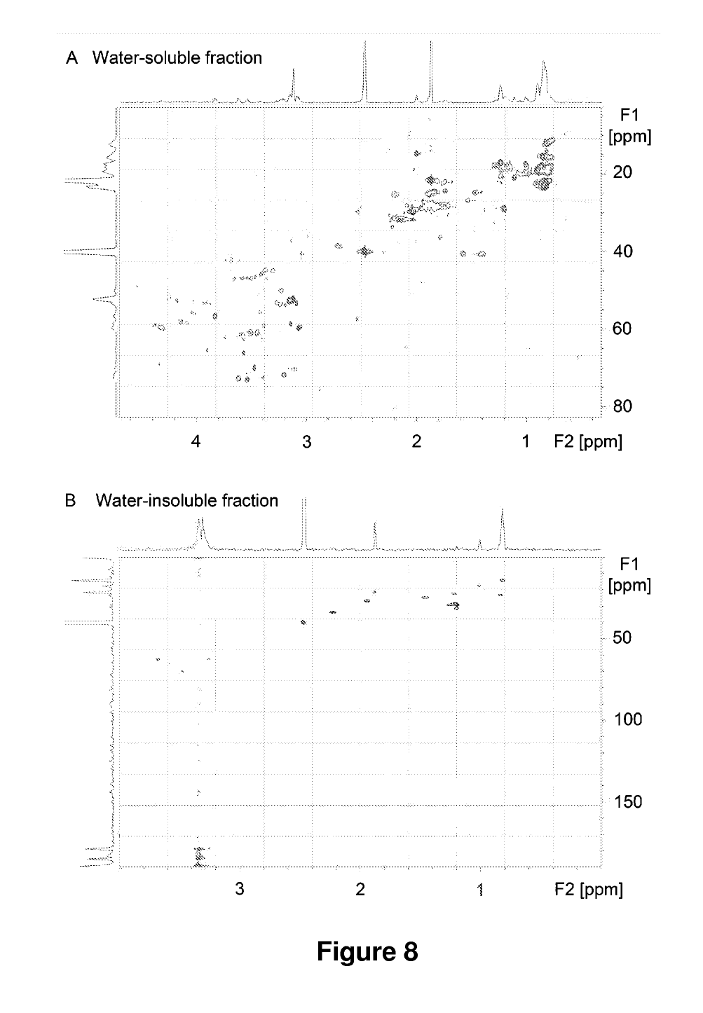

HSQC NMR spectra of the DMSO-soluble and -insoluble biofilm fractions from 1-day biofilms (FIG. 8) are typical for random coil polypeptides with the expected distribution of methyne, methylene, and methyl proton/carbon pairs along with characteristic b-CH₂ serine peaks (d_{H} 3.6-3.9 ppm, d_{C} 62-65 ppm). The spectrum for the water-soluble fraction (FIG. 8A) is more complex and appears to contain both polypeptide and saccharide peaks, consistent with the IR results. The spectrum from the water-insoluble material (FIG. 8B) is noticeably different from typical polysaccharide or nucleic acid HSQC spectra, which would be dominated by hydroxylated carbons mostly downfield of 70 ppm, a few downfield methyl groups, and in the latter case, sp² hybridized carbons.

### Biofilm-Specific Proteins in the Extracellular Matrix of NTHi Biofilms

A proteomic analysis of soluble proteins taken from the ECM of 1-day and 4-day biofilms was compared with a similar proteomic analysis of total proteins taken from planktonic bacteria. Proteins from planktonic bacteria were identified using peptide thresholds set at 80% minimum and protein thresholds set at 80% minimum with a 1-peptide minimum. These thresholds produced a list of 151 identified proteins (Tables 5-7). Proteins from the ECM of 1-day and 4-day colony biofilms were identified using peptide thresholds set at 99.9% minimum and protein thresholds set at 95% minimum with a 3-peptide minimum. All proteins common to both the planktonic and biofilm lists were eliminated. The remaining 18 proteins were designated as being biofilm-specific proteins (Table 1). Two biofilm-specific proteins were associated only with the 4-day biofilms (transketolase 2 and chaperone protein dnaK 2) and one protein (glyceraldehyde-3-phosphate dehydrogenase) was associated only with the 1-day biofilm. The remaining 15 identified proteins were present in extracellular matrix from 1- and 4-day biofilms. The majority of the proteins (7 in total) were associated with the bacterial cell membranes, while the others were either enzymes involved with metabolism, catabolism or glycolysis, or were chaperones (Table 1).

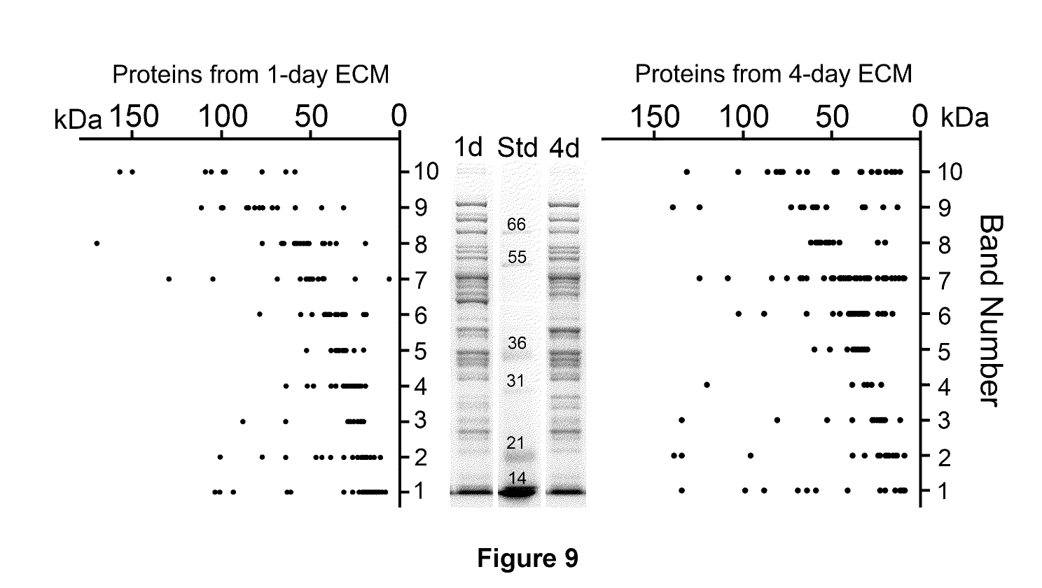

In order to determine if proteolysis was occurring in the soluble fraction of the extracellular matrix, we matched the gel slices used for the proteomic analysis with the molecular mass of the proteins detected by mass spectroscopy. The proteins from the 1-day biofilm roughly followed an expected pattern with smaller proteins being detected at the top of the gel and the larger proteins at the bottom of the gel (FIG. 9), suggesting minimal proteolysis. The 4-day biofilm proteins followed the general trend of smaller proteins at the top of the gel (FIG. 9), and proteins increasing in size toward the base of the gel. However, there were some anomalies in that some higher molecular mass proteins were detected at the top of the gel and small proteins were present at the bottom of the gel (FIG. 9). These results would suggest that large proteins are being fragmented and some low molecular mass proteins are forming aggregates or undergoing polymerization.

### Immunolocalization of DNA-Directed RNA Polymerase in the NTHi Biofilm

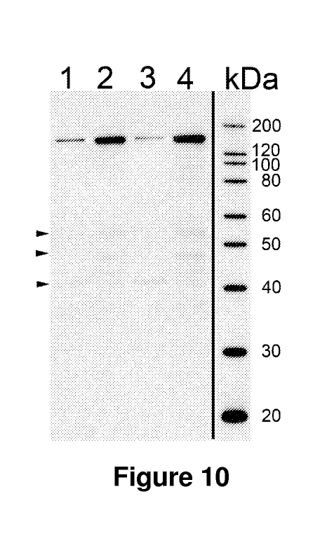

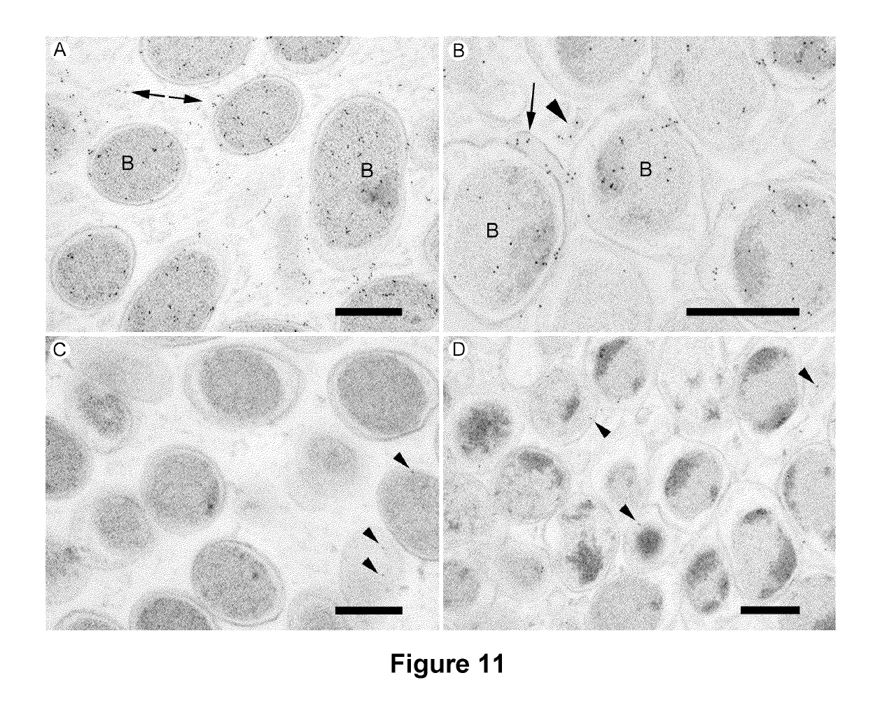

In order to determine if the proteins we had detected by proteomic analysis were in fact part of the extracellular matrix, or were from bacterial cells contaminating the soluble protein fraction, we used protein-specific antibodies to perform an immunolabeling analysis of the biofilm matrix. First, soluble protein suspensions from the ECM of colony biofilms were probed by western blotting using commercial antibodies directed against a selection of the proteins we had identified. Some of the antibodies detected more than one band on the westerns, suggesting the proteins were being degraded. However, specific anti-DNA-directed RNA polymerase bound strongly to a protein band in the 150 kDa range (where the DNA-directed RNA polymerase would be expected to appear) and showed only minimal fragmentation of the protein (FIG. 10). The anti-DNA-directed RNA polymerase antibodies bound to soluble protein taken from 1- and 4-day biofilm ECM, and 1- and 4-day biofilm pellets (FIG. 10). In order to determine if the protein was associated only with bacterial cells, or was also present in the extracellular matrix, we immunolabeled sections of cryo-prepared resin sections through 1-day and 4-day colony biofilms. The specific antibodies bound to bacterial cells in the biofilms (FIG. 11) and were also associated with the ECM (FIG. 11). Extracellular label was present in 1-day biofilms (FIG. 11A), but the labeling intensity was higher over sections through the 4-day biofilms (FIG. 11B). In the 4-day biofilms, the label was associated with the cell cytoplasm, extracellular regions of the biofilm, small extracellular vesicles, and with ECM that was in close association with the bacterial cells (FIG. 11B). Negative controls, where specific DNA-directed RNA polymerase antibodies were omitted, showed small amounts of gold labeling over the biofilm sections (FIGS. 11C & D). A more detailed and quantitative study of the labeling pattern of the anti-DNA-directed RNA polymerase antibodies and other antibodies on NTHi biofilms is in progress.

### Immunolocalization of Ornithine Carbamoyltransferase (OCT) in the NTHi Biofilm

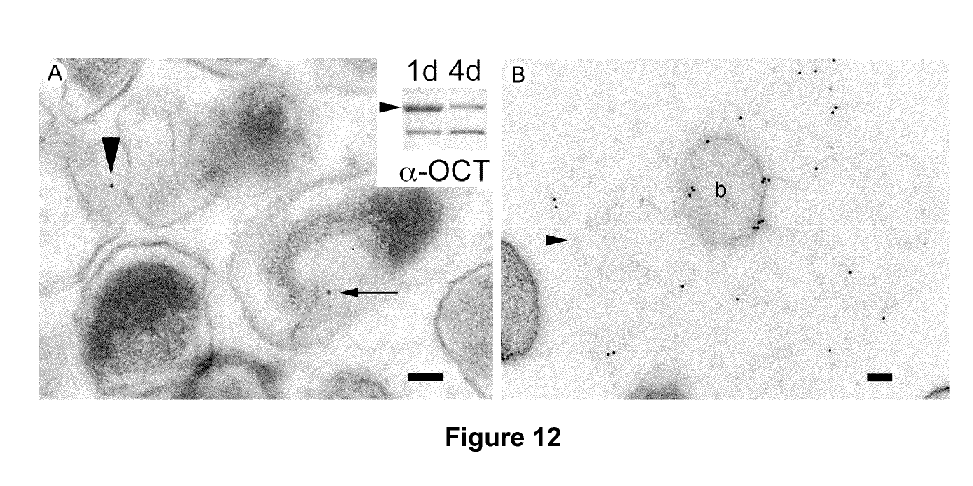

Commercial antibodies directed against ornithine carbamoyltransferase were applied to Lowicryl-embedded sections of 1-day colony biofilms to determine if the protein was in an extracellular location, as indicated by our proteomic analysis. The anti-OCT antibody showed little affinity for the colony biofilms and the small amount of immunogold labeling observed over the sections was restricted to the bacterial cell surface and intracellular regions (FIG. 12A). In order to determine if this weak anti-OCT labeling was characteristic for all NTHi biofilm formed in vitro, we applied the antibodies to sections through NTHi biofilms formed in capillary tubes. On tube biofilms, the anti-OCT labeled a small population of bacterial cells as well as large areas of extracellular material that was associated with bacterial cells (FIG. 12B).

### Involvement of Extracellular Proteins in NTHi Biofilm Formation

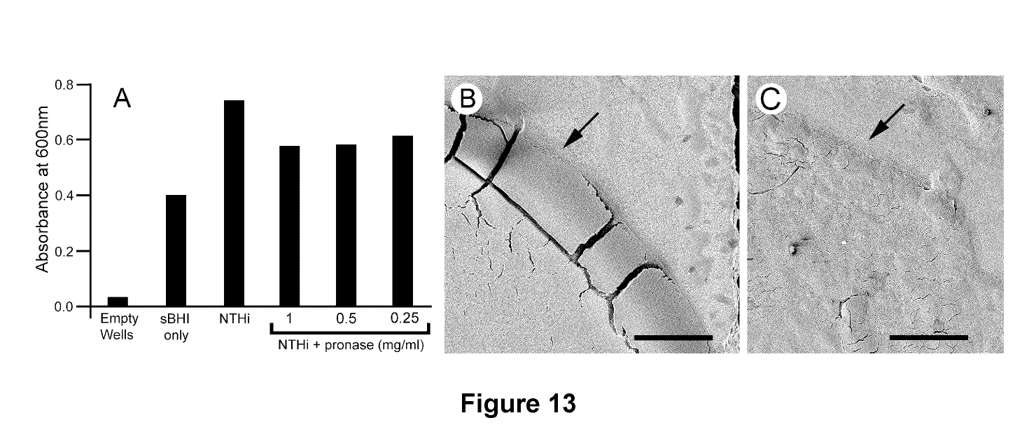

Comparing the amount of biofilm deposited on the bottom of 96-well plates using crystal violet plate assays, and grown with and without the presence of pronase demonstrated that the pronase had an inhibitory effect on the formation of NTHi biofilms (FIG. 13). The optical density after crystal violet staining for biofilms formed after one-day of incubation was higher than for similar biofilms formed in the presence of pronase (FIG. 13A). The inhibitory effect of the pronase was present at all concentrations tested (1 μg/mL, 0.5 μg/mL and 0.25 μg/mL); however, the inhibitory effect declined with decreasing pronase concentrations (FIG. 13). The OD600 of the original culture before crystal violet staining showed that exposure to pronase had little effect on bacterial growth.

SEM examination of biofilms revealed a similar inhibitory effect of pronase on biofilm formation. Biofilms formed in the absence of pronase were easily detected on the filters as disc-like structures with well-defined edges, while biofilms formed in the presence of pronase completely lacked these distinct contours (FIGS. 13B & C).

### Immunolocalization of Transketolase and Glyceraldehyde-3-Phosphate Dehydrogenase (GAPDH)

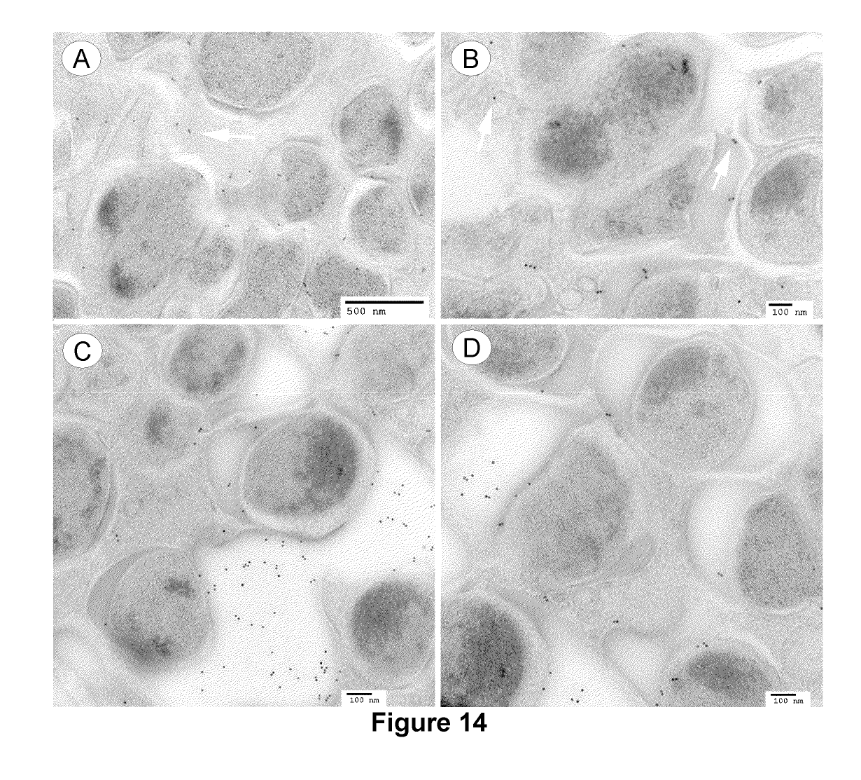

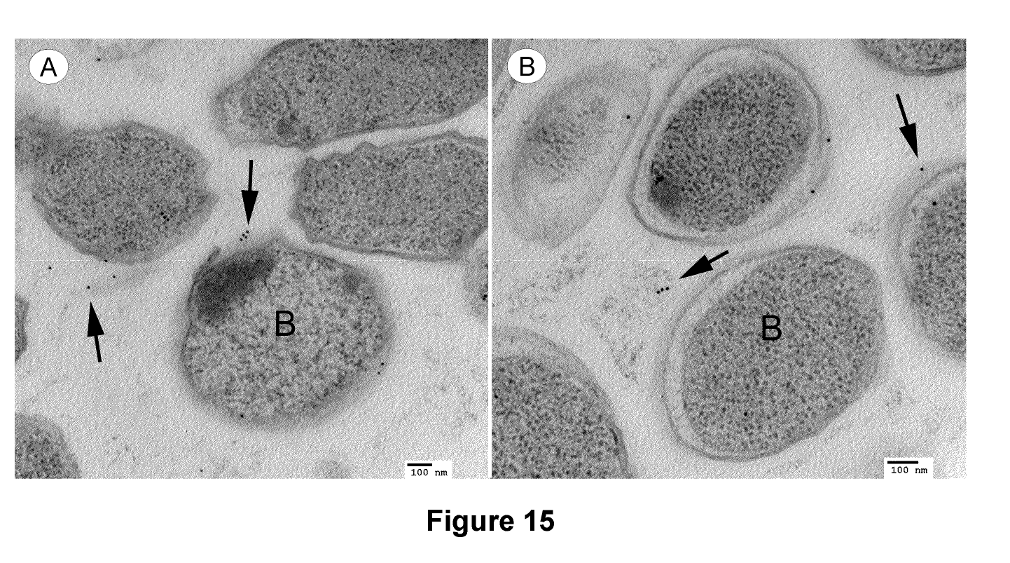

Referring to FIGS. 14 and 15, transketolase and glyceraldehyde-3-phosphate dehydrogenase (GAPDH) are found outside of the bacterial cells within the biofilm matrix. This confirms that our proteomic analysis did not pick up bacterial fragments in the soluble part of the biofilm extracellular matrix.

NTHi is a commensal organism colonizing the respiratory tract of humans, which is able to transform into a human pathogen. The diseases caused by NTHi range from the trivial to lethal, yet they all appear to be associated with biofilm formation on mucosal surfaces (Hall-Stoodley L, et al “Direct detection of bacterial biofilms on the middle-ear mucosa of children with chronic otitis media” Jama 2006, 296(2):202-211; Murphy TF et al. “Nontypeable Haemophilus influenzae as a pathogen in children” Pediatr Infect Dis J 2009, 28(1):43-48; De Schutter I, et al. “Microbiology of bronchoalveolar lavage fluid in children with acute nonresponding or recurrent community-acquired pneumonia: identification of nontypeable Haemophilus influenzae as a major pathogen” Clin Infect Dis 2011, 52(12):1437-1444; Murphy T F “Respiratory infections caused by non-typeable Haemophilus influenzae” Curr Opin Infect Dis 2003, 16(2):129-134; Penttila M, et al. “Bacterial findings in acute maxillary sinusitis—European study” Acta Otolaryngol Suppl 1997, 529:165-168; and Klein J O “Role of nontypeable Haemophilus influenzae in pediatric respiratory tract infections” Pediatr Infect Dis J 1997, 16(2 Suppl):S5-8). For NTHi, attachment to mammalian cells appears to require protein interactions (Swords W E, et al. “Non-typeable Haemophilus influenzae adhere to and invade human bronchial epithelial cells via an interaction of lipooligosaccharide with the PAF receptor” Mol Microbiol 2000, 37(1):13-27; Ahren I L, et al. “Protein D expression promotes the adherence and internalization of non-typeable Haemophilus influenzae into human monocytic cells” Microb Pathog 2001, 31(3):151-158; Ahren I L, et al. “The importance of a beta-glucan receptor in the nonopsonic entry of nontypeable Haemophilus influenzae into human monocytic and epithelial cells” J Infect Dis 2001, 184(2):150-158; Avadhanula V, et al. “Nontypeable Haemophilus influenzae adheres to intercellular adhesion molecule 1 (ICAM-1) on respiratory epithelial cells and upregulates ICAM-1 expression” Infect Immun 2006, 74(2):830-838; Berenson C S, et al. “Nontypeable Haemophilus influenzae-binding ganglio sides of human respiratory (HEp-2) cells have a requisite lacto/neolacto core structure” FEMS Immunol Med Microbiol 2005, 45(2):171-182; Bookwalter J E, et al. “A carcinoembryonic antigen-related cell adhesion molecule 1 homologue plays a pivotal role in nontypeable Haemophilus influenzae colonization of the chinchilla nasopharynx via the outer membrane protein P5-homologous adhesin” Infect Immun 2008, 76(1):48-55; Kawakami K, et al. “Attachment of nontypable Haemophilus influenzae to human pharyngeal epithelial cells mediated by a ganglioside receptor” Microbiol Immunol 1998, 42(10):697-702; and Fink D L, et al. “The Haemophilus influenzae Hap autotransporter binds to fibronectin, laminin, and collagen IV” Infect Immun 2002, 70(9):4902-4907), and perhaps the involvement of some surface structures (Novotny L A, et al. “Epitope mapping of the outer membrane protein P5-homologous fimbrin adhesin of nontypeable Haemophilus influenzae” Infect Immun 2000, 68(4):2119-2128; and Barsum W, et al. “Interaction of fimbriated and nonfimbriated strains of unencapsulated Haemophilus influenzae with human respiratory tract mucus in vitro” Eur Respir J 1995, 8(5):709-714) but not others (Clemans D L, et al. “Analysis of pilus adhesins from Haemophilus influenzae biotype IV strains” Infect Immun 2001, 69(11):7010-7019). The broad range of protein interactions reported for attachment of the bacteria to epithelial cell surfaces suggests attachment mechanisms may vary between cells at different locations within the body, or that attachment is not a simple process requiring one ligand-receptor combination. Instead, the bacteria could be using combinations of extracellular or surface proteins to attach to cells in non-specific ways.

To determine how the NTHi bacteria might use proteins to attach to surfaces, we have previously examined colony biofilms formed on abiotic filters (Webster P, et al. “Distribution of bacterial proteins in biofilms formed by non-typeable Haemophilus influenzae. J Histochem Cytochem 2006; Webster P, et al. “Ultrastructural preservation of biofilms formed by non-typeable Hemophilus influenzae” Biofilms 2004, 1:165-182; and Gallaher T K, et al. “Identification of biofilm proteins in non-typeable Haemophilus influenzae” BMC Microbiol 2006, 6:65). Watnick and Kolter (Watnick P I and Kolter R “Steps in the development of a Vibrio cholerae El Tor biofilm” Mol Microbiol 1999, 34(3):586-595) have previously shown that the morphology of colony biofilms is similar to that of biofilms formed in flow cells. Colony biofilms have become a useful tool for antibiotic testing (Anderl J N, et al. “Role of antibiotic penetration limitation in Klebsiella pneumoniae biofilm resistance to ampicillin and ciprofloxacin” Antimicrob Agents Chemother 2000, 44(7):1818-1824; and Anderl J N, et al. “Role of nutrient limitation and stationary-phase existence in Klebsiella pneumoniae biofilm resistance to ampicillin and ciprofloxacin” Antimicrob Agents Chemother 2003, 47(4):1251-1256). We chose to use colony biofilms because they mimic the biofilms that may be formed in the middle ear. During an infection, such biofilms colonize biotic surfaces within or on top of a mucous layer that covers epithelial cells. Newly forming biofilms thus have the possibility of being exposed on one surface to a non-liquid environment of humid air. Another convenient feature of colony biofilms is that they produce increased amounts of protein that can be conveniently collected, facilitating subsequent analysis. For TEM examination, high pressure freezing imposed technical restrictions on the size and shape of specimens but by forming the biofilms on filters we were able to produce biofilms that could be easily handled for ultrastructural analysis.

Ultrastructural examination of NTHi biofilms during formation revealed that as early as 1 hr after inoculation bacterial cells were covered in amorphous material, which also covered the filter substrate in regions devoid of bacteria. The early appearance of this extracellular matrix suggested that it originated from intact bacteria, not dead or dying cells. The material could be easily washed away by a protocol that included sonication, and was not easily cross-linked by aldehydes, suggesting that it could be carbohydrate in nature; however, previous studies have shown showed that proteinase K, a broad-spectrum protease, could disrupt the biofilm forming process of NTHi bacteria (Izano E A, et al. “Intercellular adhesion and biocide resistance in nontypeable Haemophilus influenzae biofilms”Microb Pathog 2009, 46(4):207-213), indicating a role for proteins in biofilm formation.

In order to obtain sufficient quantities of ECM from the NTHi biofilms, we used 1-day biofilms, which contained large numbers of bacteria as well as large amounts of ECM. However, these biofilms are still considered to be early, or immature, biofilms (Jurcisek J A and Bakaletz L O “Biofilms formed by nontypeable Haemophilus influenzae in vivo contain both double-stranded DNA and type IV pilin protein” J Bacteriol 2007, 189(10):3868-3875). As the 12 hr and 24 hr (1-day) biofilms were essentially the same when examined by electron microscopy, consisting of tightly packed bacterial cells surrounded by amorphous extracellular material, we chose the 1-day biofilms for further analysis. In the 1-day biofilms, while the extracellular material was present throughout the biofilm, it was concentrated more at the base of the biofilm. Washing away biofilms from the filter surfaces required sonication, but the 1-day biofilms could be almost completely removed from the filters, leaving only a network of fibrous material remaining on the filter surface, presumably fabricated by the 1-day biofilms.

Soluble material was also removed from 4-day biofilms, which are also considered to be immature biofilms (Jurcisek J A and Bakaletz L O “Biofilms formed by nontypeable Haemophilus influenzae in vivo contain both double-stranded DNA and type IV pilin protein” J Bacteriol 2007, 189(10):3868-3875). These biofilms were different from the 1-day biofilms, in that less biomass could be removed from the filters. However, they had similar dry weight biomass to 1-day biofilms. The reason for the difference between dry weight results and the OD₆₀₀, which showed less biomass, was that the 4-day biofilm material was less easily washed from the surface of the filter. More biofilm material remained attached to the filter surface of the 4-day biofilms, suggesting the attachment of the biofilm to the filter had changed in some way.

In order to determine if our washing protocol was causing lysis of biofilm bacteria, and thus causing their contents to contribute to the extracellular materials we were analyzing, we collected CFU data. This data produced two surprises, the first being that during biofilm formation, the numbers of culturable bacteria increased substantially between 6 hr and 12 hr. Microscopic examination of similar biofilms showed a similar increase in biomass. The second unexpected finding from the CFU data was that a substantial drop in the numbers of culturable bacteria occurred between 12 hr and 24 hr after initial inoculation of the filters. Equally unexpected was that morphologically the 12 hr and 24 hr biofilms were an almost identical mat of tightly packed bacterial cells, which did not show any evidence of bacterial cell lysis. The loss of culturable bacteria could thus be a result of bacteria switching to the biofilm phenotype and not a loss of bacterial cells by lysis. Increased numbers of bacteria by CFU counts may not be detectable if the biofilm bacteria were less able to propagate on agar plates, a common phenomenon observed for biofilm bacteria (Costerton W, et al. “The application of biofilm science to the study and control of chronic bacterial infections” J Clin Invest 2003, 112(10):1466-1477).

Measuring the OD of the washed biofilm suspensions was a rapid way to obtain a rough estimate of the total amount of material removed from the filter. These estimates showed the biofilm biomass increasing a few hours before the increase in culturable bacteria, suggesting either an increase in bacterial cell size, an increase in non-culturable bacteria, or most likely, an increase in ECM production. The OD of the washed biofilms also revealed a loss of material from the 4-day biofilm; however, dry weight analysis and SEM examination suggest that this observation was due to biofilm material remaining attached to the filter substrate after washing. The ECM from 4-day biofilms contained lower amounts of protein than what was detectable in the 1-day biofilms. One explanation for this could be that extracellular protein is being processed in some way that makes it more difficult to solublize and thus detect. The increase in binding ability we see in the 4-day biofilms may be a consequence of protein processing.

Although our interest was in the protein content of the extracellular matrix, we also evaluated a rapid way to perform a complete analysis of the contents of the washed extracellular matrix. For this analysis we applied NMR and FTIR spectroscopy to determine their use for rapid and detailed analysis of biofilm suspensions. We have successfully used FTIR spectroscopy in the past on biofilms formed by other bacteria (Baum M M, et al. “Characterization of structures in biofilms formed by a Pseudomonas fluorescens isolated from soil” BMC Microbiol 2009, 9:103); however, NMR analysis has only recently been applied to the study of components of bacterial and fungal biofilms (Seo H, et al. “Complexity of cell-cell interactions between Pseudomonas sp. AS1 and Acinetobacter oleivorans DR1: metabolic commensalism, biofilm formation and quorum quenching” Res Microbiol 2012; and Jakobsen T H, et al “Food as a source for QS inhibitors: Iberin from Horseradish Revealed as a Quorum Sensing Inhibitor of Pseudomonas aeruginosa” Appl Environ Microbiol 2011).

Infrared spectroscopy is a powerful tool for nondestructive analysis of the chemical composition of bacterial biofilms. Briefly, this technique makes use of fact that vibrational and rotational-vibrational changes within a molecule are associated with specific energies, in an analogous fashion to UV-visible spectroscopy, which probes electronic transitions within molecules. Excitation of the energy levels occurs in well-defined regions of the electromagnetic spectrum, usually in the range of 4000 to 600 cm⁻¹. The energy of the band provides key structural information and can help identify the associated functional group, while the band intensity can be correlated quantitatively to their abundance in the sample.

Using FTIR spectroscopy we were able to identify differences between the soluble fractions taken from the biofilms and the insoluble pellets, which consisted mostly of intact bacteria (Gallaher T K, et al. “Identification of biofilm proteins in non-typeable Haemophilus influenzae” BMC Microbiol 2006, 6:65). The pellets were mostly proteinaceous with no evidence of polysaccharide, as might be expected of a concentrated pellet of bacterial cells. The soluble fractions showed evidence of both protein and polysaccharide. Although there were differences between the water-soluble and water-insoluble samples, the FTIR analysis did not detect differences between samples taken from the 1-day and 4-day biofilms.

The NMR spectra supported the FTIR findings in detecting differences between the soluble and insoluble fractions, but no differences in samples taken from 1-day or 4-day biofilms. In addition to detecting the presence of polysaccharides and proteins in the water-soluble fractions, the NMR spectra also revealed the presence of small molecules and molecules with a wide size distribution. While polypeptide and saccharide profiles were repeatedly detected, nucleic acid spectra were not obvious. However, we were able to detect DNA in the soluble ECM using a specific DNA assay that we have previously used to demonstrate the presence of DNA in Pseudomonas fluorescens biofilms (Baum M M, et al. “Characterization of structures in biofilms formed by a Pseudomonas fluorescens isolated from soil” BMC Microbiol 2009, 9:103).

Estimates of DNA in the ECM of the 1-day and 4-day biofilms showed more DNA in the ECM of the 4-day biofilms, suggesting cell lysis, releasing the DNA could be occurring over time. Release of DNA into the ECM seems to be a common occurrence in bacterial biofilms (Allesen-Holm M, et al. “A characterization of DNA release in Pseudomonas aeruginosa cultures and biofilms” Mol Microbiol 2006, 59(4):1114-1128; Bockelmann U, et al. “Bacterial extracellular DNA forming a defined network-like structure” FEMS Microbiol Lett 2006, 262(1):31-38; Izano E A, et al. “Differential roles of poly-N-acetylglucosamine surface polysaccharide and extracellular DNA in Staphylococcus aureus and Staphylococcus epidermidis biofilms” Appl Environ Microbiol 2008, 74(2):470-476; Mann E E, et al. “Modulation of eDNA release and degradation affects Staphylococcus aureus biofilm maturation” PLoS One 2009, 4(6):e5822; Petersen F C, et al. “DNA binding-uptake system: a link between cell-to-cell communication and biofilm formation” J Bacteriol 2005, 187(13):4392-4400; Spoering A L and Gilmore M S “Quorum sensing and DNA release in bacterial biofilms” Curr Opin Microbiol 2006, 9(2):133-137; Steinberger R E and Holden P A “Extracellular DNA in single- and multiple-species unsaturated biofilms” Appl Environ Microbiol 2005, 71(9):5404-5410; and Flemming H C and Wingender J:The biofilm matrix” Nat Rev Microbiol 2010, 8(9):623-633), including biofilms formed by NTHi (Izano E A, et al. “Intercellular adhesion and biocide resistance in nontypeable Haemophilus influenzae biofilms”Microb Pathog 2009, 46(4):207-213; and Jurcisek J A and Bakaletz L O “Biofilms formed by nontypeable Haemophilus influenzae in vivo contain both double-stranded DNA and type IV pilin protein” J Bacteriol 2007, 189(10):3868-3875). Related studies have reported a role for nucleoid-associated proteins in NTHi biofilm formation (Goodman S D, et al. “Biofilms can be dispersed by focusing the immune system on a common family of bacterial nucleoid-associated proteins” Mucosal Immunol 2011). However, unlike our study which used biofilms formed under static in vitro conditions, the previous studies used biofilms formed in vivo, or in vitro under sheer stress conditions (Izano E A, et al. “Intercellular adhesion and biocide resistance in nontypeable Haemophilus influenzae biofilms”Microb Pathog 2009, 46(4):207-213; Jurcisek J A and Bakaletz L O “Biofilms formed by nontypeable Haemophilus influenzae in vivo contain both double-stranded DNA and type IV pilin protein” J Bacteriol 2007, 189(10):3868-3875; and Goodman S D, et al. “Biofilms can be dispersed by focusing the immune system on a common family of bacterial nucleoid-associated proteins” Mucosal Immunol 2011). The mechanism for DNA release into the ECM is not well understood, however it seems that sheer stress, which could damage bacterial cells and thus release DNA, is not likely to play an important role. The facet of biofilm formation involving DNA requires further study, especially since DNA release by other species of bacteria appears to be an event controlled by biofilm-forming bacteria (Spoering A L and Gilmore M S “Quorum sensing and DNA release in bacterial biofilms” Curr Opin Microbiol 2006, 9(2):133-137).

Our previous studies of the extracellular matrix of NTHi biofilms identified 265 proteins in the soluble extracellular matrix of newly-forming NTHi biofilms (Gallaher T K, et al. “Identification of biofilm proteins in non-typeable Haemophilus influenzae” BMC Microbiol 2006, 6:65). In that study we did not compare the protein list with similar lists from planktonic bacteria, an omission we have rectified in the current study, and even expanded. The list of 18 biofilm-specific proteins we obtained in this current study were compared with the more detailed list from our previous study (Moghaddam S J, et al. “Nontypeable Haemophilus influenzae in chronic obstructive pulmonary disease and lung cancer” Int J Chron Obstruct Pulmon Dis 2011, 6:113-123) and identified five that were common to both lists. The proteins common to both our current list and the one published previously are catabolic ornithine carbamoyltransferase, 5′-nucleotidase, purine nucleotidase, purine nucleoside phosphorylase deoD-type, transketolase 2; and high-affinity zinc uptake system protein. Identification of these proteins in the soluble ECM using different approaches further strengthens their identification as biofilm-specific proteins in NTHi biofilms.

We chose to study the DNA directed RNA-polymerase protein (rpoB) in a little more depth for a variety of reasons. First, it showed the least number of bands by western blot. The protein was also identified by LC-MS/MS in a gel band that suggested it was an intact molecule, with a molecular mass in the range of 150-155 kDa. Presence of the protein has also been previously used for the successful detection of nontuberculous bacteria in tap water and diagnostic use (Shin J H, et al. “Targeting the rpoB gene using nested PCR-restriction fragment length polymorphism for identification of nontuberculous mycobacteria in hospital tap water” J Microbiol 2008, 46(6):608-614). Deletions of other subunits of the RNA polymerase (rpoE and rpoZ) have affected biofilm formation in Streptococcus mutans and Mycobacterium smegmatis (Mathew R, et al. “Deletion of the rpoZ gene, encoding the omega subunit of RNA polymerase, results in pleiotropic surface-related phenotypes in Mycobacterium smegmatis” Microbiology 2006, 152(Pt 6):1741-1750; and Xue X, et al. “The delta subunit of RNA polymerase, RpoE, is a global modulator of Streptococcus mutans environmental adaptation” J Bacteriol 2010, 192(19):5081-5092). To our knowledge, there have been no links described between the RNA polymerase and biofilm formation by NTHi. We immunolabeled thin sections through NTHi biofilms using anti-RNA polymerase antibodies to demonstrate the presence of the protein in the ECM, and not just associated with intact cells, or cell fragments. This immunolabeling seemed to be the most definitive way of showing that the cytoplasmic protein was free within the ECM and was not being identified because of contaminating bacterial cells in the ECM fractions we analyzed. Further immunolabeling experiments will make it possible to confirm the ECM location of the other proteins we have identified in the ECM.

Outer membrane proteins (OMP P1, P2, P5) in the ECM of our NTHi biofilms, and the presence of other proteins associated with the cell membrane or periplasmic space, could simply be a result of bacterial lysis. However, the absence of OMP P6 (Webster P, et al. “Distribution of bacterial proteins in biofilms formed by non-typeable Haemophilus influenzae. J Histochem Cytochem 2006) suggests that intact membranes, which may contain OMP P6, are removed during centrifugation of our preparations. It is more probable that in collecting soluble ECM proteins we also collected small vesicles from within the biofilm. Outer membrane vesicles formed by NTHi in planktonic culture have been recently purified and subjected to a proteomic analysis (Sharpe S W, et al. “Elicitation of epithelial cell-derived immune effectors by outer membrane vesicles of nontypeable Haemophilus influenzae” Infect Immun 2011, 79(11):4361-4369). Similar vesicles in the ECM of 1-day and 4-day biofilms labeled with the anti-RNA polymerase in our studies. It is possible that these vesicles are also the source of the OMPs identified in our proteomic study. Interestingly, the three OMPs we identified in the ECM are the same three OMPs present in the purified vesicles from planktonic bacteria (Sharpe S W, et al. “Elicitation of epithelial cell-derived immune effectors by outer membrane vesicles of nontypeable Haemophilus influenzae” Infect Immun 2011, 79(11):4361-4369).

Standing alone, our results represent a list of unrelated proteins that add little to our understanding of how biofilms are formed; however, some of the proteins we identified in NTHi biofilms have been detected in other bacterial biofilm systems. Oligopeptidase A has been reported to have a general role in bacterial protein degradation (Jain R and Chan M K “Support for a potential role of E. coli oligopeptidase A in protein degradation” Biochem Biophys Res Commun 2007, 359(3):486-490). The high-affinity zinc uptake system protein (znuB) is linked to lower virulence (Campoy S, et al. “Role of the high-affinity zinc uptake znuABC system in Salmonella enterica serovar typhimurium virulence” Infect Immun 2002, 70(8):4721-4725), and intracellular growth (Kim S, et al. “Zinc uptake system (znuA locus) of Brucella abortus is essential for intracellular survival and virulence in mice” J Vet Med Sci 2004, 66(9):1059-1063). The chaperone protein DnaK 2, which is involved in protein folding of nascent polypeptides, and which requires ATP (Straus D, et al. “DnaK, DnaJ, and GrpE heat shock proteins negatively regulate heat shock gene expression by controlling the synthesis and stability of sigma 32” Genes Dev 1990, 4(12A):2202-2209; and Szabo A, et al. “The ATP hydrolysis-dependent reaction cycle of the Escherichia coli Hsp70 system DnaK, DnaJ, and GrpE”Proc Natl Acad Sci USA 1994, 91(22):10345-10349) may be required for the maintenance of drug-resistant persister cells in Escherishia coli (Hansen S, et al. “Role of global regulators and nucleotide metabolism in antibiotic tolerance in Escherichia coli” Antimicrob Agents Chemother 2008, 52(8):2718-2726). DnaK 2 may also have roles in biofilm attachment (Mangalappalli-Illathu A K and Korber D R “Adaptive resistance and differential protein expression of Salmonella enterica serovar Enteritidis biofilms exposed to benzalkonium chloride” Antimicrob Agents Chemother 2006, 50(11):3588-3596; Mangalappalli-Illathu A K and Lawrence J R, Korber D R “Cells in shearable and nonshearable regions of Salmonella enterica serovar Enteritidis biofilms are morphologically and physiologically distinct” Can J Microbiol 2009, 55(8):955-966; and Yamanaka T, et al. “Gene expression profile and pathogenicity of biofilm-forming Prevotella intermedia strain 17” BMC Microbiol 2009, 9:11) and biofilm formation (Lemos J A, et al. “Physiologic effects of forced down-regulation of dnaK and groEL expression in Streptococcus mutans” J Bacteriol 2007, 189(5):1582-1588).

Glyceraldehyde-3-phosphate dehydrogenase (GAPDH) is produced at higher levels in mature and late-stage Pseudomonas aeruginosa biofilms (Sauer K, et al. “Pseudomonas aeruginosa displays multiple phenotypes during development as a biofilm” J Bacteriol 2002, 184(4):1140-1154), and antisera to GAPDH were protective against lethal doses of Streptococcus agalactiae (Hughes M J, et al. “Identification of major outer surface proteins of Streptococcus agalactiae” Infect Immun 2002, 70(3):1254-1259). There are reports of GAPDH being down-regulated in older (5-day) P. aeruginosa biofilms (Whiteley M, et al. “Gene expression in Pseudomonas aeruginosa biofilms” Nature 2001, 413(6858):860-864). This observation fits with our study that showed a reduction in detectable GAPDH in 4-day NTHi biofilms when compared with 1-day biofilms. The sialic acid-binding periplasmic protein (siaP) may be important for NTHi biofilms because NTHi bacteria are incapable of synthesizing sialic acid. Instead, NTHi utilize siaP to scavenge free sialic acid from the host environment (Jurcisek J et al. “Role of sialic acid and complex carbohydrate biosynthesis in biofilm formation by nontypeable Haemophilus influenzae in the chinchilla middle ear.” Infect Immun 2005, 73(6):3210-3218; and Johnston J W, et al. “Characterization of the N-acetyl-5-neuraminic acid-binding site of the extracytoplasmic solute receptor (SiaP) of nontypeable Haemophilus influenzae strain 2019” J Biol Chem 2008, 283(2):855-865). Acquired sialic acid is added to LOS, which promotes biofilm formation by NTHi in vitro and bacterial persistence within the middle ear or lung in vivo (Swords W E, et al. “Sialylation of lipooligosaccharides promotes biofilm formation by nontypeable Haemophilus influenzae” Infect Immun 2004, 72(1):106-113). The sialic acid content of the LOS increases concomitantly with the transition of organisms to a biofilm form (Greiner L L, et al. “Nontypeable Haemophilus influenzae strain 2019 produces a biofilm containing N-acetylneuraminic acid that may mimic sialylated O-linked glycans” Infect Immun 2004, 72(7):4249-4260).

One protein that we found to be of particular interest in our protein list and which has been detected in other bacteria with a variety of other functions, including bacterial attachment and biofilm formation (Sauer K, et al. “Pseudomonas aeruginosa displays multiple phenotypes during development as a biofilm” J Bacteriol 2002, 184(4):1140-1154; Oosthuizen M C, et al. “Proteomic analysis reveals differential protein expression by Bacillus cereus during biofilm formation” Appl Environ Microbiol 2002, 68(6):2770-2780; Southey-Pillig C J, et al. “Characterization of temporal protein production in Pseudomonas aeruginosa biofilms” J Bacteriol 2005, 187(23):8114-8126; Lemos J A, et al. “Physiologic effects of forced down-regulation of dnaK and groEL expression in Streptococcus mutans” J Bacteriol 2007, 189(5):1582-1588; Winterhoff N, et al. “Identification and characterization of two temperature-induced surface-associated proteins of Streptococcus suis with high homologies to members of the Arginine Deiminase system of Streptococcus pyogenes” J Bacteriol 2002, 184(24):6768-6776; Alam S I, et al. “Differential proteomic analysis of Clostridium perfringens ATCC13124; identification of dominant, surface and structure associated proteins” BMC Microbiol 2009, 9:162; Hanna S L, et al. “Comparison of proteins expressed by Pseudomonas aeruginosa strains representing initial and chronic isolates from a cystic fibrosis patient: an analysis by 2-D gel electrophoresis and capillary column liquid chromatography-tandem mass spectrometry” Microbiology 2000, 146 (Pt 10):2495-2508; and Neway J O and Switzer R L “Degradation of ornithine transcarbamylase in sporulating Bacillus subtilis cells” J Bacteriol 1983, 155(2):522-530), was ornithine carbamoyltransferase (OCT). This was identified in Staphylococcus epidermidis as a fibronectin-binding broad-spectrum-adhesive protein (Hussain M, et al. “Identification and characterization of a novel 38.5-kilodalton cell surface protein of Staphylococcus aureus with extended-spectrum binding activity for extracellular matrix and plasma proteins” J Bacteriol 2001, 183(23):6778-6786). In Gram-positive bacteria, fibronectin binding proteins have been linked to pathogenesis (Schwarz-Linek U, et al. “Fibronectin-binding proteins of gram-positive cocci” Microbes Infect 2006, 8(8):2291-2298). A more well understood role for OCT in microbes is its involvement with arginine biosynthesis (Hanna S L, et al. “Comparison of proteins expressed by Pseudomonas aeruginosa strains representing initial and chronic isolates from a cystic fibrosis patient: an analysis by 2-D gel electrophoresis and capillary column liquid chromatography-tandem mass spectrometry” Microbiology 2000, 146 (Pt 10):2495-2508), catalyzing the reaction between ornithine and carbamoyl phosphate to produce citrulline and orthophosphate. Mammals use OCT as part of the urea cycle (Jackson M J, et al. “Mammalian urea cycle enzymes” Annu Rev Genet 1986, 20:431-464) and it is located in mitochondria of liver cells. Combined, these reports point to OCT being a protein with a role in metabolism and which exhibits adhesive functions. Proteins with multifunctional roles are present in eukaryotic cells (Muresan V and Muresan Z “Unconventional functions of microtubule motors” Arch Biochem Biophys 2012; Scott A, et al. “SLPI and elafin: multifunctional antiproteases of the WFDC family” Biochem Soc Trans 2011, 39(5):1437-1440; and Cassiday L A and Maher L J, 3rd “Having it both ways: transcription factors that bind DNA and RNA” Nucleic Acids Res 2002, 30(19):4118-4126) as well as bacteria (Oehmcke S, et al. “Streptococcal M proteins and their role as virulence determinants” Clin Chim Acta 2011, 411(17-18):1172-1180; and Harraghy N, et al. “The adhesive and immunomodulating properties of the multifunctional Staphylococcus aureus protein Eap” Microbiology 2003, 149(Pt 10):2701-2707).

The use of the pronase-soaked filters enabled the direct examination of the disruptive effects of pronase on biofilm formation. Pronase represents a conglomerate of proteinases, which are excreted by Streptomyces griseus, and indiscriminately denature native proteins or protein fragments (Narahashi, 1970). While pronase is not bactericidal, it appeared to prevent NTHi bacteria from attaching to one another or/and to the surface of the filter. These observations suggest a role for proteins acting as intercellular adhesins during biofilm formation by NTHi bacteria. Other researchers came to a similar conclusion using proteinase K, another broad-spectrum protease, to disrupt early biofilm formation by NTHi (Izano et al., 2009). The involvement of proteins, which mediate the adhesion of cells to abiotic surfaces, eukaryotic cells and other bacteria, is a widely observed phenomenon of a wide range of bacteria and fungi (Dunne, 2002; Gibbons, 1989; Norde & Lyklema, 1989). The extent with which matrix associated or freely diffusing proteins are actually involved in the adhesion process in NTHi as well as other bacterial biofilms, needs to be determined.

We have observed the early events of NTHi biofilm formation in vitro and documented a rapid attachment of bacteria to substrate due to production of ECM. A conservative estimate identified 18 proteins that were unique to the NTHi biofilm. These proteins were easily removed from the biofilm suggesting they might be present in fluids taken from infected regions, such as the sinus or middle ear. A logical next step, which we are taking, is to screen middle ear fluids for the presence of one or more of these proteins, with a focus on the DNA directed RNA polymerase protein. We have demonstrated that this protein is present in the ECM of the immature NTHi biofilms and is not associated with cell fragments. In addition to being possible targets for diagnostic use, the proteins present in the ECM of NTHi biofilms may have an important role in attaching bacterial cells to one another and to surfaces. Extracellular proteins may also be important components of the complex biofilm structures that microscopy is beginning to reveal in biofilms formed by other species of bacteria. It is clear that no matter what role these extracellular proteins play in biofilm formation or maintenance, it will be revealing to find how such large amounts of cytoplasmic proteins are released into the ECM.

### Preparation of Non-Typeable Haemophilus Influenzae (NTHi) Colony Biofilms

NTHi biofilms on Millipore filters were prepared as previously described (Webster P, et al. “Distribution of bacterial proteins in biofilms formed by non-typeable Haemophilus influenzae. J Histochem Cytochem 2006). Frozen aliquots of NTHi clone 2019 were thawed, plated onto chocolate agar plates and grown overnight at 37° C. in 5% CO₂ and 95% relative humidity (culture conditions applied to all bacterial and biofilm incubations). BHI broth supplemented with hemin and NAD (Poje G and Redford R J “General methods for culturing Haemophilus influenzae” In: Haemophilus influenzae protocols. Edited by Herbert M A, Hood D W, Moxon E R, vol. 71. Totowa, New Jersey: Humana Press; 2003: 51-56) was inoculated with colonies formed on the agar, and the suspension incubated overnight (overnight culture). After 20 hr relative numbers of bacteria in liquid BHI suspension were estimated by reading the OD at 600 nm (Poje G and Redford R J “General methods for culturing Haemophilus influenzae” In: Haemophilus influenzae protocols. Edited by Herbert M A, Hood D W, Moxon E R, vol. 71. Totowa, New Jersey: Humana Press; 2003: 51-56).

Overnight culture was diluted 1:200 in fresh BHI broth and inoculated onto filters (Millipore Corp., Billerica, Mass., Catalog # GSWP 025 00) on chocolate agar plates (Webster P, et al. “Distribution of bacterial proteins in biofilms formed by non-typeable Haemophilus influenzae. J Histochem Cytochem 2006; and Webster P, et al. “Ultrastructural preservation of biofilms formed by non-typeable Hemophilus influenzae” Biofilms 2004, 1:165-182). The filters with bacteria were incubated for 24 hr (1-day) or 96 hr (4-day). The NTHi colony biofilms on filters (Webster P, et al. “Ultrastructural preservation of biofilms formed by non-typeable Hemophilus influenzae” Biofilms 2004, 1:165-182; and Zahller J and Stewart P S “Transmission electron microscopic study of antibiotic action on Klebsiella pneumoniae biofilm” Antimicrob Agents Chemother 2002, 46(8):2679-2683) were either prepared for examination in the transmission electron microscope (TEM), the scanning electron microscope (SEM), proteomic analysis, or were used to estimate total numbers of viable bacteria.

### Biofilm Growth for Chemical Assay

10×20 ml BHI broth with above mentioned supplements in 50 ml centrifugation tubes were inoculated with 0.5 ml NTHi strain 2019 from a 5 ml overnight culture and incubated for one day. The supernatant was separated from the insoluble material and both fractions were dried using a speed-vac. The same protocol was followed with biofilms incubated for four days.

### Estimation of Colony-Forming Units (CFU's) and Dry Weights

Viable bacteria estimates (CFU) present in the colony biofilms were obtained using established counting methods (Herigstad B, et al. “How to optimize the drop plate method for enumerating bacteria” J Microbiol Methods 2001, 44(2):121-129). 25 filters for each time point were inoculated with overnight culture, incubated for increasing times, placed in sterile phosphate buffered saline and sonicated using a sonicator bath for 6 min. The optical density of the sonicated suspension was read at 600 nm to obtain an estimate of total biomass, and the suspension then serially diluted in sterile BHI broth. Aliquots of 5 μl were removed from each of the serial dilutions and placed onto chocolate agar plates. Estimates of total viable bacteria were calculated after 24 hr incubation and the results were tabulated as numbers of viable bacteria per filter.

### Scanning Electron Microscopy

Colony biofilms were rapidly frozen by immersion in liquid propane and subsequently stored in liquid nitrogen. Frozen specimens were freeze-substituted in dry ethanol containing 1% glutaraldehyde at −80 ° C., gradually warmed to 4° C. in 100% ethanol, and critical point dried. The dried biofilms were mounted onto specimen stubs, sputter-coated with 6 nm thick films of platinum, and examined in a scanning electron microscope (SEM, XL30 SFEG, FEI, Hillsboro, Oreg.). Biofilms grown for SEM examination were prepared on small filter strips for ease of handling. Cutting a notch in the top right corner of the filter strip facilitated identification of the inoculated side of the filter was common during the early stages of biofilm development since few traces of the bacteria were detectible on the dried filters when examined by light microscopy.

### Transmission Electron Microscopy

Colony biofilms were rapidly frozen by high-pressure freezing (EMPACT2 HPF, Leica Microsystems Inc. Deerfield, Ill.) (McDonald K L, et al. “Recent advances in high-pressure freezing: equipment- and specimen-loading methods” Methods Mol Biol 2007, 369:143-173) and freeze substituted in ethanol containing 1% osmium tetroxide. The freeze-substituted biofilms were embedded in Lowicryl HM20 (EMS, Hatfield, Pa.) at −50° C. under UV light at −50° C. (AFS2, Leica Microsystems Inc.,) (Webster P, et al. “Distribution of bacterial proteins in biofilms formed by non-typeable Haemophilus influenzae. J Histochem Cytochem 2006; and Webster P, et al. “Ultrastructural preservation of biofilms formed by non-typeable Hemophilus influenzae” Biofilms 2004, 1:165-182). Thin sections (60-80 nm) of Lowicryl-embedded biofilms were labeled with specific mouse monoclonal [8RB13] to RNA polymerase beta (Abcam, ab81865; Cambridge, Mass.) and 10 nm protein A-gold (PAG; University of Utrecht, The Netherlands) using sequential labeling protocols (Webster P, et al. “Distribution of bacterial proteins in biofilms formed by non-typeable Haemophilus influenzae. J Histochem Cytochem 2006).

Sections were imaged using a Tecnai G2 20 TEM (FEI Inc, Hillsboro, Oreg.) and images were digitally recorded (XR41, AMT, MA). When required, brightness and contrast adjustments were applied to the whole image (Adobe®Photoshop, Adobe, San Jose, Calif.).

### Crystal Violet Assay for Biofilm Formation.

Bacterial suspensions were incubated for increasing times in 96-well plates either in BHI broth without treatment or in BHI broth supplemented with pronase at dilutions ranging from 2 μg/mL to 0.25 μg/mL. The crystal violet assay plate method (Webster P, et al. “Distribution of bacterial proteins in biofilms formed by non-typeable Haemophilus influenzae. J Histochem Cytochem 2006; Murphy T F and Kirkham C “Biofilm formation by nontypeable Haemophilus influenzae: strain variability, outer membrane antigen expression and role of pili” BMC Microbiol 2002, 2:7; Webster P, et al. “Ultrastructural preservation of biofilms formed by non-typeable Hemophilus influenzae” Biofilms 2004, 1:165-182; and Watnick P I and Kolter R “Steps in the development of a Vibrio cholerae El Tor biofilm” Mol Microbiol 1999, 34(3):586-595) was used to evaluate biofilm formation. Bacterial cultures incubated overnight were exposed to a 1% solution of crystal violet solution for 15 min, washed extensively and then air-dried. The crystal violet stain remaining in the wells was resuspended in ethanol, and the absorbance (OD₆₀₀ₙₘ) read. Sterile BHI was substituted for bacterial cultures in control experiments.

### Chemical Assays, Fourier Transform Infrared Spectroscopy (FTIR), Nuclear Magnetic Resonance Spectroscopy (NMR)

Ultraviolet-visible spectra were recorded on a Model 8452A (Hewlett-Packard/Agilent Technologies, Santa Clara, Calif.) diode array spectrophotometer. Fluorescence spectra were acquired on a Model F-4500 fluorescence spectrometer (Hitachi High-Technologies, Pleasanton, Calif.). Infrared spectra were acquired on a Magna-IR 560 (Nicolet/Thermo Scientific, Madison, Wis.) FTIR spectrometer as potassium bromide discs. NMR Spectra were acquired on the DMSO-d₆ soluble fraction of the freeze-dried 4-day biofilms using an Inova 600 (Varian/Agilent Technologies, Santa Clara, Calif.) NMR spectrometer with an inverse triple resonance probe. All experiments were done with standard pulse programs and parameter sets supplied with the Chempack extension of the VnmrJ applications software (Agilent Technologies, Santa Clara, Calif.).

Chemical analysis of freeze-dried bacterial biofilms (1-day and 4-day) was carried out using the approach described previously (Baum MM, et al. “Characterization of structures in biofilms formed by a Pseudomonas fluorescens isolated from soil” BMC Microbiol 2009, 9:103). This method avoids physical and chemical disruption of the biofilms and mathematically subtracts the media contribution to the biofilm/media mixture analyzed. Total protein and nucleic acid concentration (UV absorption), total protein concentration (modified Lowry assay), and DNA concentration (DAPI conjugation, fluorometry) in the biofilms were measured using the SDS-buffer (1 mM Tris/Tris HCl, 0.1 mM EDTA, 0.15 M NaCl, 1% w/v SDS; pH 7.51 at 25° C.) soluble fractions (ca. 3 mg mL⁻¹). Total carbohydrate and uronic acid concentrations (Filisetti-Cozzi T M and Carpita N C “Measurement of uronic acids without interference from neutral sugars” Anal Biochem 1991, 197(1):157-162) were determined using freeze-dried biofilm powders suspended in deionized water. These suspensions immediately afforded clear solutions upon addition of the concentrated sulfuric acid.

### SDS-PAGE Analysis

Biofilms were dislodged from support filters by sonication, bacteria removed by centrifugation, and the supernatants subjected to an SDS-PAGE separation using a NuPAGE system and Novex bis-tris gels (Invitrogen, Carlsbad, Calif.) (Webster P, et al. “Distribution of bacterial proteins in biofilms formed by non-typeable Haemophilus influenzae. J Histochem Cytochem 2006).

### Proteomic Analysis

NTHi colony biofilms were dislodged from their filter substrates by 3 cycles of washing in PBS using an ultrasonic bath at 35 kHz (Aquasonic 75T, VWR International Inc., West Chester, Pa.) for 3 min followed by incubation on ice for another 3 minutes. The resulting slurry of bacterial biofilm material in PBS was transferred to a fresh tube. Biofilm components soluble in PBS were separated from the insoluble fractions by centrifugation at 17,000×g at 4° C. for 5 minutes.

Soluble bacterial components were separated by 1-D SDS-PAGE. Two lanes from the gel of each developmental stage were divided into 10 separate bands and digested with trypsin using established methods (Gallaher T K, et al. “Identification of biofilm proteins in non-typeable Haemophilus influenzae” BMC Microbiol 2006, 6:65). Protein digests of each band were analyzed using a nanoLC-2D system (Eksigent, Dublin, Calif.) interfaced with an autosampler and an ABI/MDS SCIEX 4000 QTRAP (ABI, Ontario, Canada). Nanoflow LC was performed using C-18 reverse phase chromatography (ZORBAX 300SB-C18) with an acetonitrile/water (containing 0.1% formate) gradient (5-85% over 75 min) at 300 nL min⁻¹. Data analysis was first performed using MASCOT software (Matrix Science, London, England), and subsequently refined using Scaffold v. 2.0 (Proteome Software, Portland, Oreg.). Final results met the major criteria outlined by Carr et al. (Carr S, et al. “The need for guidelines in publication of peptide and protein identification data: Working group on publication guidelines for peptide and protein identification data” Mol Cell Proteomics 2004, 3(6):531-533). The proteins from 1-day and 4-day biofilms were tabulated and compared with proteins identified from planktonic forms of NTHi bacteria. The molecular masses of all the biofilm proteins identified by mass spectroscopy were plotted according to their original position in the SDS-PAGE gel.

### Colony Biofilms

NTHi in liquid BHI suspension were diluted 1:200 in fresh BHI broth and innoculated onto filters (Millipore Corp., Billerica, Mass., Catalog # GSWP 025 00) on chocolate agar plates (Webster et al., 2004; Webster et al., 2006). The filters with bacteria were incubated at 37° C. in 5% CO2 and 95% relative humidity for 24 hr (1-day) or 96 hr (4-day). Some biofilms were prepared on filters that had previously been soaked with pronase (2 μg, 1 μg, 0.5 μg and 0.25 μg initial concentration), and incubated for 24 hours (1-day) or 96 hours (4-day) before being rapidly frozen and prepared for scanning electron microscopy (SEM). The NTHi colony biofilms on filters (Webster et al., 2004; Zahller & Stewart, 2002) were either prepared for examination in the transmission electron microscope (TEM), the scanning electron microscope (SEM), proteomic analysis, or were used to estimate total numbers of viable bacteria.

TABLE 1
Biofilm specific proteins identified by LC/MS/MS of soluble ECM fraction

| | UniProt | | | | | Peptide | | |
|---|---|---|---|---|---|---|---|---|
| Identified Proteins | Accession # | mol wt | Gene | Taxonomy | Location | matches | 1-Day | 4-Day |
| Biosynthesis/ | | | | | | | | |
| Metabolism/Catabolism | | | | | | | | |
| Oligopeptidase A | OPDA_HAEIN | 78 kDa | prlC | Haemophilus influenzae | (Cytoplasm, presumed) | 3 | 2 | 2 |
| Ornithine | OTCC_HAEIN | 38 kDa | arcB | Haemophilus influenzae | Cytoplasm | 4 | 2 | 1 |
| carbamoyltransferase, | | | | | | | | |
| catabolic | | | | | | | | |
| Probable 5′-nucleotidase | 5NTD_HAEIN | 66 kDa | NA | Haemophilus influenzae | (Cytoplasm, presumed) | 4 | 1 | 2 |
| Purine nucleoside | DEOD_HAEI8 | 26 kDa | deoD | Haemophilus influenzae | (Cytoplasm, presumed) | 3 | 2 | 2 |
| phosphorylase deoD-type | | | | | | | | |
| Transketolase 2 | TKT2_PASMU | 73 kDa | tktB | Pasteurella multocida | (Cytoplasm, presumed) | 4 | 0 | 1 |
| Cell membrane/ | | | | | | | | |
| transport/cell surface | | | | | | | | |
| High-affinity zinc uptake | ZNUA_HAEIN | 38 kDa | znuA | Haemophilus influenzae | Periplasm | 4 | 3 | 3 |
| system protein znuA | | | | | | | | |
| Iron-utilization | FBPA_HAEIN; | 36 kDa | fbpA | Haemophilus influenzae; | Periplasm | 3 | 4 | 5 |
| periplasmic protein | FBPA_NEIMA | | | Neisseria meningitidis | | | | |
| Outer membrane protein | OPP11_HAEIN; | 49 kDa | ompP1 | Haemophilus influenzae | Cell membrane | 4 | 3 | 2 |
| P1 | Q9KHG0_HAEIN | | | | | | | |
| Outer membrane protein | OPP2A_HAEIN; | 40 kDa | ompP2 | Haemophilus influenzae | Cell membrane | 4 | 5 | 4 |
| P2 | Q48024_HAEIN; | | | | | | | |
| | OPP25_HAEIN | | | | | | | |
| Outer membrane protein | OMP51_HAEIN | 38 kDa | ompA | Haemophilus influenzae | Cell membrane | 4 | 3 | 4 |
| P5 | | | | | | | | |
| Sialic acid-binding | SIAP_HAEIN | 37 kDa | siaP | Haemophilus influenzae | Periplasm | 4 | 5 | 4 |
| periplasmic protein siaP | | | | | | | | |
| Uncharacterized | Y362_HAEIN | 32 kDa | NA | Haemophilus influenzae | Periplasm | 4 | 2 | 2 |
| periplasmic iron-binding | | | | | | | | |
| protein HI0362 | | | | | | | | |
| Chaparone/Protein | | | | | | | | |
| folding/stress response | | | | | | | | |
| Chaperone protein | DNAK2_SYNP6 | 68 kDa | dnaK2 | Synechococcus elongatus | (Cytoplasm, presumed) | 4 | 0 | 1 |
| dnaK 2 | | | | | | | | |
| Probable FKBP-type | FKBY_HAEIN | 26 kDa | NA | Haemophilus influenzae | (Cytoplasm, presumed) | 4 | 1 | 1 |
| peptidyl-prolyl cis-trans | | | | | | | | |
| isomerase | | | | | | | | |
| Glycolysis/energy | | | | | | | | |
| metabolism | | | | | | | | |
| Dihydrolipoyl | DLDH_HAEIN | 51 kDa | pdA | Haemophilus influenzae | Cytoplasm | 4 | 1 | 1 |
| dehydrogenase | | | | | | | | |
| Glyceraldehyde-3- | G3P_STREQ; | 36 kDa | gap | Streptococcus | Cytoplasm | 4 | 2 | 0 |
| phosphate dehydrogenase | G3P_STRP1 | | | dysgalactiae; | | | | |
| | | | | Streptococcus pyogenes | | | | |
| Pyruvate kinase | KPYK_SPICI | 22 kDa | pyk | Spiroplasma citri | (Cytoplasm, presumed) | 4 | 1 | 1 |
| (Fragment) | | | | | | | | |
| Translation/Transcription | | | | | | | | |
| DNA-directed RNA | RPOB_HAEI8 | 150 kDa | rpoB | Haemophilus influenzae | (Cytoplasm, presumed) | 4 | 2 | 1 |
| polymerase subunit beta′ | | | | | | | | |

TABLE 2
Biofilm biomass, protein and DNA content of NTHi 9274 colony biofilms
Dry weight and chemical analysis results from 1-day and 4-day biofilms
on filter membranes, with a biofilm growth area of 380 mm² (mean area).

| | 1-day biofilm | 4-day biofilm |
|---|---|---|
| | Amounts (μg per biofilm) | |
| Total Biomass (dry weight) | 5,590 | 5,810 |
| Total Extractable Protein | 559 | 483 |
| Protein in soluble ECM fraction | 220 | 194 |
| DNA in ECM fraction | 6.8 | 7.6 |

TABLE 3
Principal IR-active bands in water-soluble biofilm samples.

| Band location | Band | |
|---|---|---|
| (cm⁻¹) | intensity | Assignment |
| 3433 | vs, br | vN—H (1) |
| 3320 | sh | vO—H (2); vN—H (1) |
| 3096 | w | aromatic vC—H (1); |
| | | CH₂ vC—H (1) |
| 2970 | m | N—CH₃ vC—H (1); |
| | | N—CH₂ vC—H (1); CO₂⁻ |
| | | vC═O (1); vC—N (1) |
| 2953 | m | vC—H (2-3) |
| 2900 | sh | vC—H (2-3) |
| 2648 | sh, br | vPO—H (1) |
| 1655 | vs | amide I vC═O (3-5) |
| 1590 | vs | amide II vC═O (3-5) |
| 1410 | vs | C—H bending in CH₂ (6) |
| 1252 | m | O-acetyl ester vC═O (2-3, 6); nucleic acid |
| | | vP═O (7) |
| 1160 | vs | polysaccharide vC—C and vC—O (2-4, 7) |
| 1084 | vs | polysaccharide vC—OH (2, 7) |
| 860 | s | polysaccharide vC—C, |
| | | C—C—O & C—C—H |
| | | bending (2); aromatic vC—H (1) |
| 544 | s | N/A |

vs, very strong;
s, strong,
m. medium;
w, weak;
sh, shoulder;
br, broad

TABLE 4
Principal IR-active bands in water-insoluble biofilm samples.

| Band location | Band | |
|---|---|---|
| (cm⁻¹ ) | intensity | Assignment |
| 3330 | vs | vN—H (1) |
| 3100 | sh | vO—H (2); vN—H (1) |
| 2968 | m | aromatic vC—H (1); |
| | | CH₂ vC—H (1) |
| 2933 | m | vC—H (2-3) |
| 2875 | sh | vC—H (2-3) |
| 1675 | vs | amide I vC═O (3-5) |
| 1552 | vs | amide II vC═O (3-5) |
| 1493 | m | CO₂⁻ vC═O (1); |
| | | aromatic C—C (1) |
| 1404 | m | C—H bending in CH₂ (6) |
| 1247 | vs | O-acetyl ester vC═O (2-3, 6); |
| | | nucleic acid vP═O (7) |
| 1088 | vs | polysaccharide vC—OH (2, 7) |
| 995 | w | vC—O—H (1); vP—N (1) |
| 957 | w | vC═C (1); vP—N (1) |
| 661 | sh | N/A |
| 554 | m | N/A |

vs, very strong;
s, strong,
m. medium;
w, weak;
sh, shoulder;
br, broad

TABLE 5
Proteins detected in planktonic bacteria only

| | | | | | | | |
|---|---|---|---|---|---|---|---|
| | UniProt | | | | | | |
| Identified protein | Accession # | mol wt | Gene | Taxonomy | 1-d | 4-d | Pl |

| | | | | | |
|---|---|---|---|---|---|
| 1-phosphofructokinase | K1PF_XANCP | 33 kDa | fruK | Xanthomonas campestris | 2 |
| 2-(5″-triphosphoribosyl)-3′-dephosphocoenzyme-A | CITG_ECOL5 | 32 kDa | citG | Escherichia coli | 2 |
| synthase | | | | | |
| 3-oxoacyl-[acyl-carrier-protein] reductase | FABG_HAEIN | 26 kDa | fabG | Haemophilus influenzae | 1 |
| 30S ribosomal protein S1 | RS1_HAEIN | 60 kDa | rpsA | Haemophilus influenzae | 1 |
| 30S ribosomal protein S2 | RS2_CAUCR | 29 kDa | rpsB | Caulobacter crescentus | 1 |
| 30S ribosomal protein S6 | RS6_LEGPA | 13 kDa | rpsF | Legionella pneumophila str. | 1 |
| | | | | Paris | |
| 50S ribosomal protein L15 | RL15_HAEI8 | 15 kDa | rplO | Haemophilus influenzae | 2 |
| 50S ribosomal protein L3 | RL3_HAEIN; | 22 kDa | rplC | Haemophilus influenzae; | 5 |
| | RL3_BUCAI; | | | Buchnera aphidicola | |
| | RL3_HAEI8 | | | | |
| 60 kDa chaperonin 2 | CH602_MESSB; | 58 kDa | groL2 | Mesorhizobium sp.; | 2 |
| | CH602_RHORT | | | Rhodospirillum rubrum | |
| ABC transporter, ATP-binding and permease protein | Q6MTE9_MYCMS | 51 kDa | abc | Mycoplasma mycoides | 1 |
| Acetyl-/propionyl-coenzyme A carboxylase alpha chain | BCCA_MYCLE | 64 kDa | bccA | Mycobacterium leprae | 1 |
| Acetyl-coenzyme A carboxylase carboxyl transferase | ACCA_BACCR | 36 kDa | accA | Bacillus cereus | 1 |
| subunit alpha | | | | | |
| Acetylglutamate kinase | ARGB_DESDG | 32 kDa | argB | Desulfovibrio desulfuricans | 1 |
| Adenylate kinase | KAD_TREPA | 23 kDa | adk | Treponema pallidum | 1 |
| Agmatinase | SPEB_PHOLL | 34 kDa | speB | Photorhabdus luminescens | 1 |
| Alkanesulfonate monooxygenase | SSUD_XANAC | 42 kDa | ssuD | Xanthomonas axonopodis | 1 |
| Aspartate ammonia-lyase | ASPA_HAEIN | 51 kDa | aspA | Haemophilus influenzae | 1 |
| B Chain B, Structure Of E. Coli Uridine | UDP_ECOLI | 27 kDa | udp | Escherichia coli | 2 |
| Phosphorylase At 2.0 a | | | | | |
| Bifunctional protein hldE | HLDE_PSEPF | 50 kDa | hldE | Pseudomonas fluorescens | 1 |
| Carbamoyl-phosphate synthase large chain | CARB_RHILO | 126 kDa | carB | Rhizobium loti | 1 |
| Chaperone protein dnaJ | DNAJ_YERPE | 41 kDa | dnaJ | Yersinia pestis | 1 |
| Chaperone surA | SURA_VIBCH | 48 kDa | surA | Vibrio cholerae | 1 |
| Chaperone surA homolog | SURA_HAEIN | 35 kDa | surA | Haemophilus influenzae | 1 |
| Chorismate synthase | AROC_NITEU | 42 kDa | aroC | Nitrosomonas europaea | 1 |
| CPG DNA methylase | MTSI_SPISQ | 44 kDa | sssIM | Spiroplasma monobiae | 2 |
| Cytochrome c-552 | NRFA_HAEI8 | 60 kDa | nrfA | Haemophilus influenzae | 2 |
| Cytochrome c3, 50 kDa (Fragment) | CYC35_DESAC | 3 kDa | * | Desulfuromonas acetoxidans | 2 |
| Dissimilatory sulfite reductase beta subunit | Q6V1Z9_9BACT | 29 kDa | dsrB | unclutured bacterium | 1 |
| DNA gyrase subunit B | GYRB_MYCCT | 72 kDa | gyrB | Mycoplasma capricolum | 1 |
| DNA polymerase III polC-type | DPO3_MYCPU | 166 kDa | polC | Mycoplasma pulmonis | 1 |
| DNA translocase ftsK | FTSK_HELPY | 96 kDa | ftsK | Helicobacter pylori | 2 |
| Electron transport complex protein rnfE | RNFE_BUCAI | 25 kDa | rnfE | Buchnera aphidicola | 1 |
| Elongation factor P | EFP_HAEIN | 21 kDa | efp | Haemophilus influenzae | 1 |
| Elongation factor Tu-B | EFTU2_YERPE | 43 kDa | tufB | Yersinia pestis | 1 |
| Exoglucanase/xylanase | GUX_CELFI | 51 kDa | cex | Cellulomonas fimi | 2 |
| Exopolysaccharide production negative regulator | EXOR_RHILV | 29 kDa | exoR | Rhizobium leguminosarum | 1 |
| Glutaminyl-tRNA synthetase | SYQ_HAEI8 | 64 kDa | glnS | Haemophilus influenzae | 1 |
| Glutamyl-tRNA(Gln) amidotransferase subunit A | GATA_STAA3 | 53 kDa | gatA | Staphylococcus aureus | 1 |
| Holliday junction ATP-dependent DNA helicase | RUVA_HAEI8 | 23 kDa | ruvA | Haemophilus influenzae | 1 |
| ruvA | | | | | |
| Inositol-1-monophosphatase | SUHB_HAEIN | 30 kDa | suhB | Haemophilus influenzae | 1 |
| Leucyl/phenylalanyl-tRNA--protein transferase | LFTR_NITOC | 28 kDa | aat | Nitrosococcus oceani ATCC | 1 |
| | | | | 19707 | |
| Lipoprotein-releasing system transmembrane protein | LOLC_BUCAP | 46 kDa | lolC | Buchnera aphidicola | 1 |
| lolC | | | | | |
| Monofunctional biosynthetic peptidoglycan | MTGA_PSE14 | 27 kDa | mtgA | Pseudomonas syringae | 1 |
| transglycosylase | | | | | |
| N-acetylmuramoyl-L-alanine amidase cwlM | CWLM_BACLI | 28 kDa | cwlM | Bacillus licheniformis | 1 |
| N-succinylglutamate 5-semialdehyde dehydrogenase | ASTD_CHRSD | 52 kDa | astD | Chromohalobacter salexigens | 1 |
| Na+/H+ antiporter | Q8YZ31_ANASP | 77 kDa | * | Anabaena sp. | 1 |
| NADH-quinone oxidoreductase subunit D | NUOD_MYCPA | 48 kDa | nuoD | Mycobacterium | 1 |
| | | | | paratuberculosis | |
| NADPH-dependent 7-cyano-7-deazaguanine | QUEF_CHRVO | 31 kDa | queF | Chromobacterium violaceum | 8 |
| reductase | | | | | |
| Nisin biosynthesis protein nisB | NISB_LACLA | 118 kDa | nisB | Lactococcus lactis | 1 |
| Nitrate/nitrite response regulator protein homolog | NARP_HAEIN | 23 kDa | narP | Haemophilus influenzae | 1 |
| Oligo-1,6-glucosidase | O16G_BACCE | 66 kDa | malL | Bacillus cereus | 2 |
| Oligoribonuclease | ORN_MANSM | 21 kDa | orn | Mannheimia | 1 |
| | | | | succiniciproducens | |
| Oxytetracycline exporter | Q7BTG4_STRRM | 56 kDa | otrB | Streptomyces rimosus | 1 |
| Peptidyl-tRNA hydrolase 1 | PTH1_CORJK | 21 kDa | pth1 | Corynebacterium jeikeium | 1 |
| | | | | K411 | |
| Phosphate import ATP-binding protein pstB 2 | PSTB2_VIBVY | 31 kDa | pstB2 | Vibrio vulnificus | 3 |
| Phosphoenolpyruvate-protein phosphotransferase | PT1_BUCAI | 64 kDa | ptsI | Buchnera aphidicola | 1 |
| Phospholipase C | PHLC1_CLOPE | 46 kDa | plc | Clostridium perfringens | 2 |
| Phosphonates import ATP-binding protein phnC 1 | PHNC1_ANASP | 29 kDa | phnC1 | Anabaena sp. | 1 |
| Photosystem I assembly protein ycf3 | YCF3_PROMP | 20 kDa | ycf3 | Prochlorococcus marinus | 3 |
| Pilus assembly protein | Q989D4_RHILO | 43 kDa | * | Rhizobium loti | 1 |
| Probable 5′-nucleotidase | 5NTD_HAEIN | 66 kDa | * | Haemophilus influenzae | 1 |
| Probable cell wall amidase lytH | LYTH_STAEQ | 33 kDa | lytH | Staphylococcus epidermidis | 1 |
| | | | | RP62A | |
| Probable cystathionine gamma-synthase (Fragment) | METB_HERAU | 35 kDa | metB | Herpetosiphon aurantiacus | 1 |
| Probable outer membrane drug efflux lipoprotein | Q7NRE4_CHRVO | 50 kDa | * | Chromobacterium violaceum | 2 |
| Probable periplasmic serine protease do/hhoA-like | HTOA_HAEIN | 49 kDa | * | Haemophilus influenzae | 2 |
| Probable phosphomannomutase | Y740_HAEIN | 53 kDa | * | Haemophilus influenzae | 3 |
| Propionicin-F | PRONF_PROFF | 28 kDa | pcfA | Propionibacterium | 1 |
| | | | | freudenreichii | |
| Protein recA | RECA_MYCLE | 78 kDa | recA | Mycobacterium leprae | 5 |
| Putative mating pair formation protein | Q847E3_PSEPU | 38 kDa | mpfI | Pseudomonas putida | 1 |
| Putative NADH dehydrogenase/NAD(P)H | HADB_BURPI | 21 kDa | hadB | Ralstonia pickettii | 1 |
| nitroreductase hadB | | | | | |
| Putative pbp5 repressor PsR | Q9X4R1_ENTFC | 29 kDa | psR | Enterococcus faecium | 1 |
| Putative peptidase B | PEPB_HAEIN | 47 kDa | pepB | Haemophilus influenzae | 1 |
| Putative uncharacterized protein | Q6ALP8_DESPS | 16 kDa | * | Desulfotalea psychrophila | 2 |
| Putative uncharacterized protein | Q8CWR6_STRR6 | 50 kDa | * | Streptococcus pneumoniae | 1 |
| Putative uncharacterized protein | Q6MLS0_BDEBA | 63 kDa | * | Bdellovibrio bacteriovorus | 1 |
| Riboflavin biosynthesis protein ribAB | RIBAB_CHLPN | 46 kDa | ribBA | Chlamydia pneumoniae | 1 |
| RuBisCO operon transcriptional regulator | RBCR_THIFE | 34 kDa | rbcR | Thiobacillus ferrooxidans | 1 |
| S-layer protein | SLAP_CAMFE | 96 kDa | sapA | Campylobacter fetus | 1 |
| Sensor protein | Q7M9K7_WOLSU | 43 kDa | * | Wolinella succinogenes | 1 |
| Shikimate dehydrogenase | AROE_NEIPH | 29 kDa | aroE | Neisseria pharyngis | 1 |
| Streptothricin acetyltransferase | STA_STRLA | 20 kDa | sta | Streptomyces lavendulae | 2 |
| Stringent starvation protein A homolog | SSPA_HAEIN | 24 kDa | sspA | Haemophilus influenzae | 1 |
| Thiamine biosynthesis protein thiC 2 | THIC2_GEOMG | 47 kDa | thiC2 | Geobacter metallireducens GS- | 1 |
| | | | | 15 | |
| Transposase for insertion sequence element IS231C | T231C_BACTB | 56 kDa | * | Bacillus thuringiensis | 1 |
| tRNA pseudouridine synthase A | TRUA_STRAW | 31 kDa | truA | Streptomyces avermitilis | 1 |
| UDP-N-acetylglucosamine--N-acetylmuramyl- | MURG2_BACCR | 39 kDa | murG2 | Bacillus cereus | 1 |
| (pentapeptide) pyrophosphoryl-undecaprenol N- | | | | | |
| acetylglucosamine transferase 2 | | | | | |
| UDP-N-acetylmuramate--L-alanine ligase | MURC_COXBU | 51 kDa | murC | Coxiella burnetii | 1 |
| Uncharacterized HIT-like protein BUsg_345 | YHIT_BUCAP | 13 kDa | * | Buchnera aphidicola | 5 |
| Uncharacterized periplasmic iron-binding protein | Y362_HAEIN | 32 kDa | * | Haemophilus influenzae | 1 |
| HI0362 | | | | | |
| Uncharacterized protein HI1048 | Y1048_HAEIN | 41 kDa | * | Haemophilus influenzae | 1 |
| Uncharacterized protein HI1349 | Y1349_HAEIN | 18 kDa | * | Haemophilus influenzae | 1 |
| Uncharacterized protein HI1427 | Y1427_HAEIN | 31 kDa | * | Haemophilus influenzae | 1 |
| Uncharacterized protein HI1624 | Y1624_HAEIN | 26 kDa | * | Haemophilus influenzae | 1 |
| Uncharacterized protein Rv2850c/MT2916 | Y2850_MYCTU | 67 kDa | * | Mycobacterium tuberculosis | 5 |
| Uncharacterized protein y4bG | Y4BG_RHISN | 30 kDa | * | Rhizobium sp. | 1 |
| Uncharacterized protein ydbJ | YDBJ_ECOL6 | 9 kDa | ydbJ | Escherichia coli O6 | 2 |
| Uncharacterized symporter yihO | YIHO_SALTY | 52 kDa | yihO | Salmonella typhimurium | 1 |
| UPF0082 protein Cgl1663/cg1872 | Y1663_CORGL | 27 kDa | * | Corynebacterium glutamicum | 2 |
| UPF0082 protein Daro_4067 | Y4067_DECAR | 26 kDa | * | Dechloromonas aromatica | 1 |
| | | | | RCB | |
| UPF0082 protein PBPRB1582 | Y5582_PHOPR | 27 kDa | * | Photobacterium profundum | 1 |
| UPF0234 protein HI1034 | Y1034_HAEIN | 19 kDa | * | Haemophilus influenzae | 1 |
| UPF0241 protein plu0229 | Y229_PHOLL | 19 kDa | * | Photorhabdus luminescens | 1 |
| UPF0246 protein Bpro_3713 | Y3713_POLSJ | 29 kDa | * | Polaromonas sp. | 1 |
| Urease subunit alpha (Fragment) | URE23_HELMU | 24 kDa | ureA | Helicobacter mustelae | 1 |
| Zinc import ATP-binding protein znuC | ZNUC_BUCAP | 27 kDa | znuC | Buchnera aphidicola | 1 |

TABLE 6
Proteins identified in planktonic bacteria and 1-day biofilms

| | UniProt | | | | | | |
|---|---|---|---|---|---|---|---|
| Identified protein | Accession # | mol wt | Gene | Taxonomy | 1-d | 4-d | Pl |
| 30S ribosomal protein S4 | RS4_GEOMG; | 24 kDa | rpsD | Geobacter metallireducens; | 1 | | 2 |
| | RS4_HAEDU | | | Haemophilus ducreyi | | | |
| 30S ribosomal protein S7 | RS7_HAEI8; | 18 kDa | rpsG | Haemophilus influenzae; | 3 | | 2 |
| | RS7_ECO57 | | | Escherichia coli | | | |
| 5-methyltetrahydropteroyltriglutamate-- | METE_HAEIN; | 85 kDa | metE | Haemophilus influenzae; | 3 | | 2 |
| homocysteine methyltransferase | METE_THEMA | | | Thermotoga maritima | | | |
| 50S ribosomal protein L11 | RL11_HAEI8; | 15 kDa | rplK | Haemophilus influenzae; | 2 | | 4 |
| | RL11_CLOP1; | | | Clostridium perfringens; | | | |
| | RL11_HAEDU; | | | Haemophilus ducreyi; | | | |
| | RL11_LACPL | | | Lactobacillus plantarum | | | |
| 50S ribosomal protein L9 | RL9_HAEIN; | 16 kDa | rplI | Haemophilus influenzae; | 3 | | 2 |
| | RL9_LEGPA; | | | Legionella pneumophila; | | | |
| | RL9_NITWN | | | Nitrobacter winogradskyi | | | |
| 6-phosphogluconate dehydrogenase | 6PGD_HAEIN; | 47 kDa | gnd | Haemophilus influenzae; | 4 | | 2 |
| | Q5MBK1_PASHA | | | Pasteurella haemolytica | | | |
| Adenylate kinase | KAD_HAEIN | 24 kDa | adk | Haemophilus influenzae | 2 | | 2 |
| Alanyl-tRNA synthetase | SYA_HAEI8; | 97 kDa | alaS | Haemophilus influenzae; | 4 | | 2 |
| | SYA_MYCS5; | | | Borrelia burgdorferi; | | | |
| | SYA_BORBU; | | | Corynebacterium jeikeium; | | | |
| | SYA_CORJK; | | | Mycoplasma synoviae | | | |
| Aspartyl-tRNA synthetase 2 | SYD2_STRMU | 67 kDa | aspS2 | Streptococcus mutans | 1 | | 3 |
| Chaperone protein htpG | HTPG_ACTAC; | 71 kDa | htpG | Aggregatibacter | 1 | | 2 |
| | HTPG_THIDA | | | actinomycetemcomitans; | | | |
| | | | | Thiobacillus denitrificans | | | |
| CtaK | Q5MD28_9DELT | 34 kDa | ctaK | Cystobacter fuscus | 1 | | 3 |
| Cysteinyl-tRNA synthetase | SYC_SYNSC; | 54 kDa | cysS | Ehrlichia canis; Hahella | 1 | | 4 |
| | SYC_EHRCJ; | | | chejuensis; Synechococcus sp. | | | |
| | SYC_HAHCH | | | | | | |
| Cytochrome c biogenesis ATP-binding | CCMA_RICCN; | 21 kDa | ccmA | Rickettsia conorii; Wolbachia | 1 | | 2 |
| export protein ccmA | CCMA_WOLPM | | | pipientis | | | |
| D-alanine--D-alanine ligase | DDL_BORBR | 34 kDa | ddl | Bordetella bronchiseptica | 1 | | 6 |
| Elongation factor Tu | EFTU_APPPP; | 44 kDa | tuf | Campylobacter jejuni; | 4 | | 2 |
| | EFTU_CAMJE; | | | Candidatus Phytoplasma; | | | |
| | EFTU_LACLA | | | Lactococcus lactis | | | |
| Formate acetyltransferase | PFLB_HAEIN | 86 kDa | pflB | Haemophilus influenzae | 2 | | 3 |
| Fumarate reductase flavoprotein subunit | FRDA_HAEIN | 66 kDa | frdA | Haemophilus influenzae | 2 | | 4 |
| Glutathione synthetase | GSHB_BRAJA | 35 kDa | gshB | Bradyrhizobium japonicum | 1 | | 2 |
| Glycerol-3-phosphate dehydrogenase | GPDA_FUSNN; | 36 kDa | gpsA | Fusobacterium nucleatum; | 1 | | 2 |
| [NAD(P)+] | GPDA_VIBPA | | | Vibrio parahaemolyticus | | | |
| GMP synthase [glutamine-hydrolyzing] | GUAA_BORGA; | 59 kDa | guaA | Borrelia garinii; Ehrlichia | 2 | | 5 |
| | GUAA_EHRRG; | | | ruminantium; Escherichia coli; | | | |
| | GUAA_ECO57; | | | Lactococcus lactis; | | | |
| | GUAA_LACLA; | | | Propionibacterium acnes | | | |
| | GUAA_PROAC | | | | | | |
| GTP-binding protein engA | ENGA_VIBPA; | 56 kDa | engA | Rickettsia prowazekii; Vibrio | 1 | | 2 |
| | ENGA_RICPR | | | parahaemolyticus | | | |
| HTH-type transcriptional regulator gntR | GNTR_ECOL6 | 36 kDa | gntR | Escherichia coli O6 | 2 | | 3 |
| Low affinity potassium transport | KUP_WIGBR | 73 kDa | kup | Wigglesworthia glossinidia | 1 | | 2 |
| system protein kup | | | | | | | |
| Phosphoglycerate kinase | PGK_HAEIN; | 41 kDa | pgk | Haemophilus influenzae; | 3 | | 4 |
| | PGK_LACDE; | | | Lactobacillus delbrueckii; | | | |
| | PGK_PASMU | | | Pasteurella multocida | | | |
| Prolyl-tRNA synthetase | SYP_PROM9; | 68 kDa | proS | Haemophilus influenzae; | 1 | | 4 |
| | SYP_BACHD; | | | Bacillus halodurans; | | | |
| | SYP_HAEI8 | | | Prochlorococcus marinus | | | |
| Putative DNA polymerase III, | Q6LN39_PHOPR; | 130 kDa | dnaE | Mycobacterium leprae; | 3 | | 2 |
| alpha subunit | DPO3A_MYCLE | | | Photobacterium profundum | | | |
| Pyridoxamine kinase | PDXY_HAEIN | 32 kDa | pdxY | Haemophilus influenzae | 1 | | 2 |
| Rhamnulokinase | RHAB_OCEIH | 53 kDa | rhaB | Oceanobacillus iheyensis | 2 | | 2 |
| Similar to ABC transporter (Permease) | Q97M26_CLOAB | 51 kDa | * | Clostridium acetobutylicum | 2 | | 1 |
| tRNA pseudouridine synthase B | TRUB_CLOPE; | 33 kDa | truB | Clostridium perfringens; | 1 | | 2 |
| | TRUB_PSYAR | | | Psychrobacter arcticum | | | |
| UPF0343 protein DVU_2892 | Y2892_DESVH; | 30 kDa | folE2 | Desulfovibrio vulgaris; | 1 | | 3 |
| | Y803_NEIMB | | | Neisseria meningitidis | | | |

TABLE 7
Proteins identified in planktonic bacteria and 4-day biofilms

| | | | | | | | |
|---|---|---|---|---|---|---|---|
| | UniProt | | | | | | |
| Identified protein | Accession # | mol wt | Gene | Taxonomy | 1-d | 4-d | Pl |

| | | | | | | |
|---|---|---|---|---|---|---|
| Aspartate aminotransferase | AAT_HAEIN | 44 kDa | aspC | Haemophilus influenzae | 1 | 2 |
| ATP synthase subunit beta | ATPB_SACD2 | 51 kDa | atpD | Saccharophagus degradans | 1 | 1 |
| Cytochrome P450-pinF1, plant-inducible | CPXC_AGRTU | 48 kDa | cyp103 | Agrobacterium tumefaciens | 1 | 6 |
| Diaminobutyrate--2-oxoglutarate aminotransferase | DAT_HAEIN | 49 kDa | dat | Haemophilus influenzae | 1 | 2 |
| Formate--tetrahydrofolate ligase | FTHS_MYCPE | 60 kDa | fhs | Mycoplasma penetrans | 1 | 3 |
| Formate-dependent nitrite reductase complex | NRFG_ECO57 | 23 kDa | nrfG | Escherichia coli | 1 | 2 |
| subunit nrfG | | | | | | |
| Modification methylase AluI | MTA1_CELCE | 59 kDa | aluIM | Cellulosimicrobium cellulans | 1 | 3 |
| N utilization substance protein B homolog | NUSB_GEOMG; | 15 kDa | nusB | Geobacter metallireducens; | 1 | 7 |
| | NUSB_TREPA | | | Treponema pallidum | | |
| Probable cysteine desulfurase | CSD_STAAM | 46 kDa | csd | Staphylococcus aureus | 2 | 8 |
| Ribosomal RNA large subunit methyltransferase H | Y4808_PSEPK; | 18 kDa | rlmH | Fusobacterium nucleatum; | 1 | 2 |
| | Y463_FUSNN | | | Pseudomonas putida | | |
| RNA polymerase sigma factor rpoD | RPOD_HELPJ; | 79 kDa | rpoD | Helicobacter pylori; Xylella | 1 | 5 |
| | RPOD_XYLFA | | | fastidiosa | | |
| Seryl-tRNA synthetase | SYS_SYNP7; | 49 kDa | serS | Haemophilus influenzae; | 2 | 8 |
| | SYS_FRATT; | | | Francisella tularensis; | | |
| | SYS_HAEIN; | | | Synechococcus elongatus; | | |
| | SYS_WOLSU | | | Wolinella succinogenes | | |
| Thiamine biosynthesis protein thiC | THIC_SHEON; | 80 kDa | thiC | Shewanella oneidensis; | 1 | 1 |
| | THIC_STRAW | | | Streptomyces avermitilis | | |
| tRNA uridine 5-carboxymethylaminomethyl | GIDA_MYCPN | 68 kDa | mnmG | Mycoplasma pneumoniae | 1 | 2 |
| modification enzyme gidA (Glucose-inhibited | | | | | | |
| division protein A) | | | | | | |
| UvrABC system protein B | UVRB_CHLTE; | 78 kDa | uvrB | Chlorobaculum tepidum; | 2 | 2 |
| | UVRB_MYCMO; | | | Mycoplasma mobile; | | |
| | UVRB_PARUW | | | Protochlamydia amoebophila | | |

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

1. A method of identifying from a sample the presence of a biofilm that comprises non-typeable Haemophilus influenza (NTHi) comprising screening the sample for binding of one or more soluble biofilm-specific proteins to antibodies or fragments thereof that specifically bind to said one or more soluble biofilm-specific proteins, wherein at least one of said one or more soluble biofilm-specific proteins is selected from the group consisting of SEQ ID NO:17, SEQ ID NO:20, SEQ ID NO:21, and SEQ ID NO:22, wherein binding to said antibodies or fragments thereof indicates the presence of a biofilm that comprises NTHi.

2. (canceled)

3. A method for diagnosing an NTHi biofilm-related disease in a subject comprising obtaining a sample from said subject and screening said sample according to the method of claim 1.

4. The method of claim 3, wherein said NTHi biofilm-related disease comprises infection by NTHi in the respiratory tract of said subject.

5. The method of claim 4, wherein said infection by NTHi is in the upper respiratory tract of said subject.

6. The method of claim 3, wherein said NTHi biofilm-related disease comprises infection by NTHi in the ear of said subject.

7. The method of claim 3, wherein said disease is Otitis media (OM).

8. The method of claim 3, wherein the sample is middle ear fluid, saliva or blood.

9. (canceled)

10. A protein microarray for screening soluble biofilm-specific proteins in a sample, comprising a substrate having attached thereto one or more antibodies or fragments thereof, said one or more antibodies or fragments thereof being specific for respective said soluble biofilm-specific proteins, wherein at least one of said soluble biofilm-specific proteins is selected from the group consisting of SEQ ID NO:17, SEQ ID NO:20, SEQ ID NO:21, and SEQ ID NO:22.

11. (canceled)

12. A formulation comprising one or more soluble biofilm-specific proteins or fragments thereof, wherein at least one of the one or more soluble biofilm-specific proteins is selected from the group consisting of SEQ ID NO:17, SEQ ID NO:20, SEQ ID NO:21, and SEQ ID NO:22.

13. The formulation of claim 12, further comprising one or more adjuvant(s).

14. A method for inducing an immune response in a patient in need thereof against a biofilm-related infection, comprising administering to the patient the formulation of claim 12.

15. The method of claim 1, wherein said antibodies comprise monoclonal antibodies, polyclonal antibodies, chimeric antibodies, humanized antibodies, or single-chain antibodies, or a combination thereof.

16. The method of claim 1, wherein the binding is detected through an assay selected from the group consisting of Western blot assay, ELISA, radioimmunoassay, immunohistochemical assay and immnuoprecipitation assay.

17. The method of claim 1, wherein said one or more soluble biofilm-specific proteins comprise a protein as set forth in SEQ ID NO:17.

18. The method of claim 1, wherein said one or more soluble biofilm-specific proteins comprise a protein as set forth in SEQ ID NO:20.

19. The method of claim 1, wherein said one or more soluble biofilm-specific proteins comprise a protein as set forth in SEQ ID NO:21.

20. The method of claim 1, wherein said one or more soluble biofilm-specific proteins comprise a protein as set forth in SEQ ID NO:22.

21. A method comprising:
diagnosing a subject with an NTHi biofilm-related disease according to the method of claim 3; and
administering to the subject a pharmaceutical composition specifically targeting the NTHi biofilm.

22. The method of claim 21, wherein the pharmaceutical composition comprises antibodies or fragments thereof, wherein the antibodies or fragments thereof specifically bind to at least one soluble biofilm-specific protein selected from the group consisting of SEQ ID NO:17, SEQ ID NO:20, SEQ ID NO:21, and SEQ ID NO:22.
